(19) United States
(12) Patent Application Publication
(10) Pub. No.: US 2024/0180809 A1
(43) Pub. Date: Jun. 6, 2024

(54) METHOD FOR DYEING KERATIN MATERIAL, INCLUDING THE USE OF AN ORGANIC C1-C6 ALKOXY SILANE, A DYEING COMPOUND, AND A HEAT TREATMENT

(71) Applicant: Henkel AG & Co. KGaA, Duesseldorf (DE)

(72) Inventors: Gabriele WESER, Essen (DE); Claudia KOLONKO, Remscheid (DE); Ulrike SCHUMACHER, Duesseldorf (DE); Caroline KRIENER, Duesseldorf (DE); Imme BREUER, Duesseldorf (DE); Angela MUELLER, Grevenbroich (DE); Bernhard BANOWSKI, Duesseldorf (DE); Jing HODES, Hagen (DE); Marcus CLAAS, Hilden (DE)

(21) Appl. No.: 18/283,471

(22) PCT Filed: Feb. 15, 2022
(86) PCT No.: PCT/EP2022/053617

§ 371 Date: Sep. 22, 2023

(30) Foreign Application Priority Data
Apr. 13, 2021 (DE) 10 2021 203 614.7

(51) Int. Cl. A61K 8/58; A45D 19/00; A61Q 5/10

(52) U.S. Cl. CPC A61K 8/585; A45D 19/0066; A45D 19/012; A61Q 5/10; A61K 2800/43; A61K 2800/432

(57) ABSTRACT

The present invention relates to a method for dyeing keratin materials, in particular human hair, comprising:
applying a composition (A) to the keratin material, the composition (A) containing one or more organic C₁-C₆ alkoxy silanes (A1) and/or the condensation products thereof, and
applying a composition (B) to the keratin material, the composition (B) containing one or more dyeing compounds (B1) from the group of the pigments and/or the direct dyes, and
a heat treatment of the keratin material.

### CROSS-REFERENCE TO RELATED APPLICATIONS

The present application is a national stage entry according to 35 U.S.C. § 371 of PCT application No.: PCT/EP2022/053617 filed on Feb. 15, 2022; which claims priority to German patent application 10 2021 203 614.7 filed on Apr. 13, 2021; all of which are incorporated herein by reference in their entirety and for all purposes.

### TECHNICAL FIELD

The present application is in the field of cosmetics and relates to a method for treating keratin material, in particular human hair, which comprises the use of two compositions (A) and (B). The composition (A) is a preparation which contains at least one organic C₁-C₆ alkoxy silane, and the composition (B) contains at least one dyeing compound from the group of the pigments and the direct dyes. Furthermore, the method comprises a heat treatment of the keratin material.

### BACKGROUND

Changing the shape and color of keratinous fibers, in particular hair, represents an important area of modern cosmetics. To change the hair color, a person skilled in the art is familiar with a variety of dyeing systems depending on the dyeing requirements. Oxidation dyes are typically used for permanent, intense dyeing with good fastness properties and good gray coverage. Such dyes typically contain oxidation dye precursors, known as developer components, and coupler components, which together form the actual dyes under the influence of oxidizing agents, for example hydrogen peroxide. Oxidation dyes are characterized by very long-lasting color results.

When using direct dyes, dyes that are already fully formed diffuse from the dye into the hair fiber. In comparison with oxidative hair coloring, the colors obtained with direct dyes have a lower durability and a more rapid washing out. Dyeing with direct dyes usually remains on the hair for a period of between 5 and 20 hair washes.

For short-term color changes on the hair and/or the skin, the use of color pigments is known. Color pigments are generally understood to mean insoluble dyeing substances. These are present in the form of small particles in the dye formulation and are deposited only from the outside onto the hair fibers and/or the skin surface. Therefore, they can generally be removed again without residue by a few washes using surfactant-containing cleaning agents. Various products of this type are available on the market under the name “hair mascara”.

EP 2168633 B1 addresses the task of producing long-lasting hair dyes using pigments. The document teaches that, when using a combination of pigment, organic silicon compound, hydrophobic polymer and a solvent, dyeing can be produced on hair which is particularly resistant to being shampooed.

The organic silicon compounds used in EP 2168633 B1 are reactive compounds from the class of alkoxy silanes. These alkoxy silanes hydrolyze at high speed in the presence of water and form—depending on the amounts of alkoxy silane and water used in each case—hydrolysis products and/or condensation products. The influence of the amount of water used in this reaction on the properties of the hydrolysis or condensation product is described, for example, in WO 2013068979 A2.

When these alkoxy silanes or their hydrolysis or condensation products are applied to keratin material, a film or a coating forms on the keratin material, which completely envelops the keratin material and in this way greatly influences the properties of the keratin material. Possible fields of application are, for example, permanent styling or also the permanent shape change of keratin fibers. In this case, the keratin fibers are mechanically brought into the desired shape and are then fixed in this form by forming the above-described coating. A further very particularly suitable application possibility is the dyeing of keratin material. In the context of this application, the coating or the film is produced in the presence of a dyeing compound, for example a pigment. The film dyed by the pigment remains on the keratin material or the keratin fibers and results in surprisingly wash-resistant dyeing.

The great advantage of the dyeing principle based on alkoxy silanes is that the high reactivity of this compound class enables very rapid coating. Thus, good dyeing results can be achieved even after brief application periods of only a few minutes. In addition, the coating is produced on the surface of the keratin material and does not alter the structure in the interior of said keratin, so that this dyeing technology represents a very gentle method of changing the coloring of the keratin material.

However, dyeing methods which have recourse to the formation of colored films or coatings are still in need of optimization. In particular, the color intensities and the fastness properties of the dyes obtained with this dyeing system can still be further improved. The hair feel and the chroma or the vibrancy of the dyes obtained with this system also still require optimization.

### SUMMARY

It was therefore the object of the present application to find a method for dyeing keratin materials, in particular human hair, which has improved color intensities and improved fastness properties, in particular improved fastness to washing and improved fastness to rubbing. Furthermore, the formulations applied in the context of this method should lead to an improved hair feel, and the dyes obtained using this method should have a particularly high vibrancy (or a particularly high chroma value).

Surprisingly, it has been found that this object can be achieved in full when the keratin material is treated in a process in which two compositions (A) and (B) are applied to the keratin material, and the keratin material undergoes heat treatment. The first composition (A) contains at least one organic C₁-C₆ alkoxy silane (A1) and/or the condensation product thereof, and the second composition (B) is characterized by its content of at least one dyeing compound from the group of the pigments and the direct dyes (B1).

The present invention firstly relates to a method for dyeing keratin material, in particular human hair, comprising
applying a composition (A) to the keratin material, the composition (A) containing one or more organic C₁-C₆ alkoxy silanes (A1) and/or the condensation products thereof, and
applying a composition (B) to the keratin material, the composition (B) containing one or more dyeing compounds (B1) from the group of the pigments and/or the direct dyes, and
a heat treatment of the keratin material.

### DETAILED DESCRIPTION

### Dyeing of Keratin Material

Keratin material is understood to mean hair, the skin, the nails (for example fingernails and/or toenails). Furthermore, wool, furs and feathers also fall under the definition of keratin material.

Keratin material is preferably understood to be human hair, human skin and human nails, in particular fingernails and toenails. Keratin material is very particularly preferably understood to mean human hair.

Organic C₁-C₆ Alkoxy Silanes (A1) and/or the Condensation Products Thereof in the Composition (A)

The composition (A) is characterized in that it contains one or more organic C₁-C₆ alkoxy silanes (A1) and/or the condensation products thereof.

The organic C₁-C₆ alkoxy silane(s) are organic, non-polymeric silicon compounds, preferably selected from the group of silanes having one, two or three silicon atoms

Organic silicon compounds, which are alternatively also referred to as organosilicon compounds, are compounds which either have a direct silicon-carbon bond (Si—C) or in which the carbon is linked to the silicon atom via an oxygen, nitrogen or sulfur atom. The organic silicon compounds according to the invention are preferably compounds which contain one to three silicon atoms. The organic silicon compounds particularly preferably contain one or two silicon atoms.

According to the IUPAC rules, the designation “silane” denotes a substance group of chemical compounds based on a silicon backbone and hydrogen. In the case of organic silanes, the hydrogen atoms are replaced, completely or in part, by organic groups such as (substituted) alkyl groups and/or alkoxy groups.

Characteristic for the C₁-C₆ alkoxy silanes according to the invention is that at least one C₁-C₆ alkoxy group is present directly bound to a silicon atom. The C₁-C₆ alkoxy silanes according to the invention thus comprise at least one structural unit R′R″R′″Si—O—(C₁-C₆ alkyl), the groups R′, R″ and R′″ representing the three other binding valencies of the silicon atom.

The or these C₁-C₆ alkoxy groups bonded to the silicon atom are very reactive and are hydrolyzed at high speed in the presence of water, the reaction rate inter alia also depending on the number of hydrolyzable groups per molecule. If the hydrolyzable C₁-C₆ alkoxy group is an ethoxy group, the organic silicon compound thus preferably contains a structural unit R′R″R′″Si—OCH2-CH3. The groups R′, R″ and R′″ again represent the three remaining free valencies of the silicon atom.

Even the addition of small amounts of water initially leads to hydrolysis and then to a condensation reaction of the organic alkoxy silanes with one another. For this reason, both the organic alkoxy silanes (A1) and the condensation products thereof can be present in the composition.

A condensation product is understood to mean a product which results by reaction of at least two organic C₁-C₆ alkoxy silanes with elimination of water and/or with elimination of a C₁-C₆ alkanol.

The condensation products can be, for example, dimers, but also trimers or oligomers, the condensation products being in equilibrium with the monomers.

Depending on the amount of water used or consumed in the hydrolysis, the equilibrium of monomeric C₁-C₆ alkoxy silane to condensation product shifts.

In the context of a very particularly preferred embodiment, a method according to the invention is characterized in that the composition (A) comprises one or more organic C₁-C₆ alkoxy silanes (A1) selected from silanes having one, two or three silicon atoms, the organic silicon compound also comprising one or more basic chemical functions.

This basic group may be, for example, an amino group, an alkylamino group or a dialkylamino group, which is preferably bound to a silicon atom via a linker. Preferably, the basic group is an amino group, a C₁-C₆ alkyl amino group or a Di(C₁-C₆) alkyl amino group.

A very particularly preferred method according to the invention is characterized in that the composition (A) contains one or more organic C₁-C₆ alkoxy silanes (A1) selected from the group of silanes having one, two or three silicon atoms, and the C₁-C₆ alkoxy silanes further comprising one or more basic chemical functions.

Very particularly good results were able to be obtained when C₁-C₆ alkoxy silanes of the formula (S-I) and/or (S-II) were used in the method according to the invention. Since, as already described above, hydrolysis/condensation already occurs in the case of traces of moisture, the condensation products of C₁-C₆ alkoxy silanes of the formula (S-I) and/or (S-II) are also covered by this embodiment.

In a further very particularly preferred embodiment, a method according to the invention is characterized in that the first composition (A) contains one or more organic C₁-C₆ alkoxy silanes (A1) of formula (S-I) and/or (S-II),

R₁R₂N-L-Si(OR₃)ₐ(R₄)_{b} (S-I)

in which
R₁, R₂ represent, independently of one another, a hydrogen atom or a C₁-C₆ alkyl group,
L represents a linear or branched, divalent C₁-C₂₀ alkylene group,
R₃, R₄ represent, independently of one another, a C₁-C₆ alkyl group,
a represents an integer from 1 to 3, and
b represents the integer 3-a, and

(R₅O)_{c}(R₆)_{d}Si-(A)ₑ-[NR₇-(A′)]_{f}-[O-(A″)]_{g}-[NR₈-(A′″)]ₕ-Si(R₆′)_{d′}(OR₅′)_{c′} (S-II),

in which
R5, R5′, R5″, R6, R6′ and R6″ represent, independently of one another, a C₁-C₆ alkyl group,
A, A′, A″, A′″ and A″″ represent, independently of one another, a linear or branched, divalent C₁-C₂₀ alkylene group,
R₇ and Rs represent, independently of one another, a hydrogen atom, a C₁-C₆ alkyl group, a hydroxy C₁-C₆ alkyl group, a C₂-C₆ alkenyl group, an amino C₁-C₆ alkyl group, or a group of formula (S-III),

(A″″)-Si(R₆″)_{d}″(OR₅″)_{c}″ (S-III),
c, represents an integer from 1 to 3,
d represents the integer 3-c,
c′ represents an integer from 1 to 3,
d′ represents the integer 3-c′,
c″ represents an integer from 1 to 3,
d″ represents the integer 3-c″,
e represents 0 or 1,
f represents 0 or 1,
g represents 0 or 1,
h represents 0 or 1,
with the proviso that at least one of the groups e, f, g and h is different from 0, and/or the condensation products thereof.

The substituents R₁, R₂, R₃, R₄, R₅′, R₅″, R₅″, R₆, R₆′, R₆″, R₇, R₈, L, A, A′, A″, A′″ and A″″ in the compounds of the formula (S-I) and (S-II) are explained by way of example below:

Examples of a C₁-C₆ alkyl group are the methyl, ethyl, propyl, isopropyl, n-butyl, s-butyl and t-butyl, n-pentyl and n-hexyl groups. Propyl, ethyl and methyl are preferred alkyl groups. Examples of a C₂-C₆ alkenyl group are vinyl, allyl, but-2-enyl, but-3-enyl and isobutenyl, preferred C₂-C₆ alkenyl groups are vinyl and allyl. Preferred examples of a hydroxy-C₁-C₆ alkyl group are a hydroxymethyl, a 2-hydroxyethyl, a 2-hydroxypropyl, a 3-hydroxypropyl, a 4-hydroxybutyl group, a 5-hydroxypentyl and a 6-hydroxyhexyl group; a 2-hydroxyethyl group is particularly preferred. Examples of an amino-C₁-C₆ alkyl group are the aminomethyl group, the 2-aminoethyl group, the 3-aminopropyl group. The 2-aminoethyl group is particularly preferred. Examples of a linear divalent C₁-C₂₀ alkylene group are, for example, the methylene group (—CH₂-—, the ethylene group (—CH₂—CH₂—), the propylene group (—CH₂—CH₂—CH₂—) and the butylene group (—CH₂—CH₂—CH₂—CH₂—). The propylene group (—CH₂—CH₂—CH₂—) is particularly preferred. Above a chain length of 3 C atoms, divalent alkylene groups may also be branched. Examples of branched, divalent C₃-C₂₀ alkylene groups are (—CH₂—CH(CH₃)—) and (—CH₂—CH(CH₃)—CH₂—).

In the organic silicon compounds of formula (S-I),

R₁R₂N-L-Si(OR₃)ₐ(R₄)_{b} (S-I),

the groups R₁ and R₂ represent, independently of one another, a hydrogen atom or a C₁-C₆ alkyl group. Very particularly preferably, the groups R₁ and R₂ both represent a hydrogen atom.

The structural unit or the linker -L-, which represents a linear or branched, divalent C₁-C₂₀ alkylene group, is located in the middle part of the organic silicon compound. The divalent C₁-C₂₀ alkylene group can alternatively also be designated a divalent C₁-C₂₀ alkylene group, which means that each -L- grouping can enter into two bonds.

Preferably, -L- represents a linear, divalent C₁-C₂₀ alkylene group. Further preferably, -L- represents a linear divalent C₁-C₆ alkylene group. Particularly preferably, -L- represents a methylene group (—CH₂—), an ethylene group (—CH₂—CH₂—), a propylene group (—CH₂—CH₂—CH₂—) or a butylene group (—CH₂—CH₂—CH₂—CH₂—). Very particularly preferably, L represents a propylene group (—CH₂—CH₂—CH₂—).

The organic silicon compounds of the formula (S-I) according to the invention,

R₁R₂N-L-Si(OR₃)ₐ(R₄)_{b} (S-I),

each bear one end of the silicon-containing grouping —Si(OR₃)a(R₄)b.

In the terminal structural unit —Si(OR₃)a(R₄)b, the groups R₃ and R₄ represent, independently of one another, a C₁-C₆ alkyl group; particularly preferably R₃ and R₄ represent, independently of one another, a methyl group or an ethyl group.

In this case, a represents an integer from 1 to 3, and b represents the integer 3-a. If a represents the number 3, then b is equal to 0. If a represents the number 2, then b is equal to 1. If a represents the number 1, then b is equal to 2.

A keratin treatment agent having particularly good properties was able to be produced when the composition (A) contains at least one organic C₁-C₆ alkoxy silane of the formula (S-I), in which the groups R₃, R₄ represent, independently of one another, a methyl group or an ethyl group.

Furthermore, dyes having the best wash fastness could be obtained when the composition (A) contains at least one organic C₁-C₆ alkoxy silane of the formula (S-I), in which the group a represents the number 3. In this case, the group b represents the number 0.

In a further preferred embodiment, a method according to the invention is characterized in that the composition (A) comprises one or more organic C₁-C₆ alkoxy silanes of the formula (S-I),

in which
R₃, R₄ represent, independently of one another, a methyl group or an ethyl group, and
a represents the number 3, and
b represents the number 0.

In a further preferred embodiment, a method according to the invention is characterized in that the composition (A) contains at least one or more organic C₁-C₆ alkoxy silanes of the formula (S-I),

R₁R₂N-L-Si(OR₃)ₐ(R₄)_{b} (S-I),

in which
R₁, R₂ both represent a hydrogen atom, and
L represents a linear, divalent C₁-C₆ alkylene group, preferably a propylene group (—CH₂—CH₂—CH₂—) or an ethylene group (—CH₂—CH₂—),
R₃ represents an ethyl group or a methyl group,
R₄ represents a methyl group or an ethyl group,
a represents the number 3, and
b represents the number 0.

Organic silica compounds of formula (I) that are particularly well suited for achieving the object according to the invention are

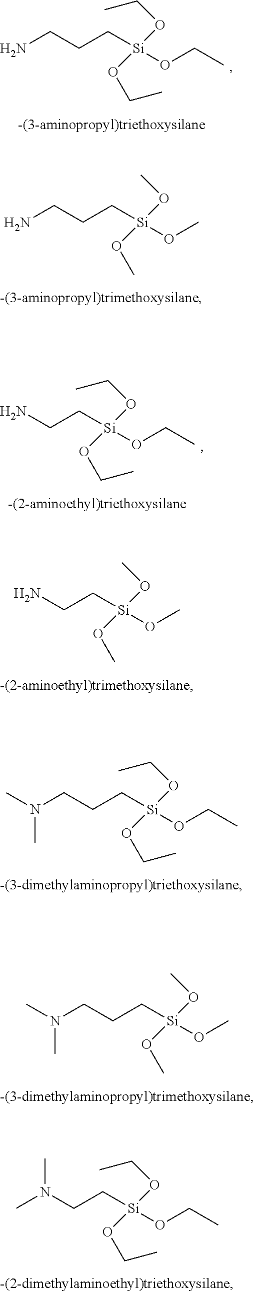

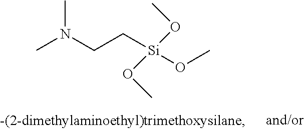

In a further preferred embodiment, a method according to the invention is characterized in that the composition (A) contains at least one organic C₁-C₆ alkoxy silane (A1) of formula (S-I), which is selected from the group consisting of
(3-aminopropyl)triethoxysilane,
(3-aminopropyl)trimethoxysilane,
(2-aminoethyl)triethoxysilane,
(2-aminoethyl)trimethoxysilane,
(3-dimethylaminopropyl)triethoxysilane,
(3-dimethylaminopropyl)trimethoxysilane,
(2-dimethylaminoethyl)triethoxysilane,
(2-dimethylaminopropyl)trimethoxysilane,
   and/or the condensation products thereof.

The aforementioned organic silicon compounds of the formula (I) are commercially available.

(3-aminopropyl)trimethoxysilane can be purchased from Sigma-Aldrich, for example. (3-aminopropyl)triethoxysilane is commercially available from Sigma-Aldrich.

In the context of a further embodiment of the method according to the invention, the composition (A) can also contain one or more organic C₁-C₆ alkoxy silanes of formula (S-II),

(R₅O)_{c}(R₆)_{d}Si-(A)ₑ-[NR₇-(A′)]_{f}-[O-(A″)]_{g}-[NR₈-(A′″)]ₕ-Si(R₆′)_{d′}(OR₅′)_{c′} (S-II),

The organosilicon compounds of the formula (S-II) according to the invention each bear, at their two ends, the silicon-containing groupings (R₅O)_{c}(R₆)dSi— and —Si(R₆′)_{d′}(OR₅′)_{c′}.

The groupings -(A)ₑ- and —[NR₇-(A′)]_{f}- and —[O-(A″)]_{g}- and —[NR₈-(A′″)]ₕ-. are located in the middle part of the molecule of the formula (S-II). In this case, each of the groups e, f, g and h can represent, independently of one another, the number 0 or 1, there being the proviso that at least one of the groups e, f, g and h is different from 0. In other words, an organic silicon compound of formula (II) according to the invention contains at least one grouping from the group consisting of -(A)- and —[NR₇-(A′)]- and —[O-(A″)]- and —[NR₈-(A′″)]-.

In the two terminal structural units (R₅O)_{c}(R₆)_{d}Si— and —Si(R₆′)_{d′}(OR₅′)_{c′}, the groups R₅, R₅′, R₅″ represent, independently of one another, a C₁-C₆ alkyl group. The groups R₆, R₆′ and R₆″ represent, independently of one another, a C₁-C₆ alkyl group.

In this case, c represents an integer from 1 to 3, and d represents the integer 3-c. If c represents the number 3, then d is equal to 0. If c represents the number 2, then d is equal to 1. If c represents the number 1, then d is equal to 2.

Similarly, c′ represents an integer from 1 to 3, and d′ represents the integer 3-c′. If c′ represents the number 3, then d′ is equal to 0. If c′ represents the number 2, then d′ is equal to 1. If c′ represents the number 1, then d′ is equal to 2.

Dyes having the best fastness to washing were able to be obtained when the groups c and c′ both represent the number 3. In this case, d and d′ both represent the number 0.

In a further preferred embodiment, a method according to the invention is characterized in that the composition (A) contains one or more organic C₁-C₆ alkoxy silanes of formula (S-II),

(R₅O)_{c}(R₆)_{d}Si-(A)ₑ-[NR₇-(A′)]_{f}-[O-(A″)]_{g}-[NR₈-(A′″)]ₕ-Si(R₆′)_{d′}(OR₅′)_{c′} (S-II),

in which
R₅ and R₅′ represent, independently of one another, a methyl group or an ethyl group,
c and c′ both represent the number 3, and
d and d′ both represent the number 0.

If c and c′ both represent the number 3 and d and d′ both represent the number 0, the organic silicon compound according to the invention corresponds to the formula (S-IIa)

(R₅O)₃Si-(A)ₑ-[NR₇-(A′)]_{f}[O-(A″)]_{g}-[NR₈-(A′″)]ₕ-Si(OR₅′)₃ (S-IIa).

The groups e, f, g and h can represent, independently of one another, the number 0 or 1, at least one group from e, f, g and h being different from zero. The abbreviations e, f, g and h accordingly define which of the groupings -(A)ₑ- and —[NR₇-(A′)]_{f}- and —-[O-(A″)]_{g}- and —[NR₈-(A′″)]ₕ- are located in the middle part of the organic silicon compound of formula (II).

In this context, the presence of certain groupings has proven to be particularly advantageous with regard to achieving wash-resistant dyeing results. Particularly good results could be obtained if at least two of the groups e, f, g and h represent the number 1. Very particularly preferably, e and f both represent the number 1. Furthermore, g and h very particularly preferably both represent the number 0.

If e and f both represent the number 1, and g and h both represent the number 0, the organic silicon compound according to the invention corresponds to the formula (S-IIb)

(R₅O)_{c}(R₆)dSi-(A)-[NR₇-(A′)]-Si(R₆′)_{d′}(OR₅′)_{c′} (S-IIb).

The groups A, A′, A″, A′″ and A″″ represent, independently of one another, a linear or branched, divalent C₁-C₂₀ alkylene group. The groups A, A′, A″, A′″ and A″″ preferably represent, independently of one another, a linear, divalent C₁-C₂₀ alkylene group. More preferably, the groups A, A′, A″, A′″ and A″″ represent, independently of one another, a linear divalent C₁-C₆ alkylene group.

The divalent C₁-C₂₀ alkylene group can alternatively also be designated a divalent C₁-C₂₀ alkylene group, which means that each grouping A, A′, A″, A′″ and A″″ can enter two bonds.

Particularly preferably, the groups A, A′, A″, A′″ and A″″ represent, independently of one another, a methylene group (—CH₂—), an ethylene group (—CH₂—CH₂—), a propylene group (—CH₂—CH₂—CH₂—) or a butylene group (—CH₂—CH₂—CH₂—CH₂—). Very particularly preferably, the groups A, A′, A″, A″″ and A″″ represent a propylene group (—CH₂—CH₂—CH₂—).

If the group f represents the number 1, then the organic silicon compound of the formula (II) according to the invention contains a structural grouping —[NR₇-(A′)]-.

If the group h represents the number 1, the organic silicon compound of the formula (II) according to the invention contains a structural grouping —[NR₈-(A′″)]-.

In this case, the groups R₇ and Rs represent, independently of one another, a hydrogen atom, a C₁-C₆ alkyl group, a hydroxy C₁-C₆ alkyl group, a C₂-C₆ alkenyl group, an amino C₁-C₆ alkyl group or a grouping of formula (S-III)

(A″″)-Si(R₆″)_{d}″(OR₅″)_{c}″ (S-III),

Very particularly preferably, the groups R₇ and R₈ represent, independently of one another, a hydrogen atom, a methyl group, a 2-hydroxyethyl group, a 2-alkenyl group, a 2-aminoethyl group or a grouping of the formula (S-III).

If the group f represents the number 1 and the group h represents the number 0, the organic silicon compound according to the invention contains the grouping [NR₇-(A′)], but not the grouping —[NR₈-(A′″)]. If the group R₇ now represents a grouping of the formula (III), the organic silicon compound thus comprises 3 reactive silane groups.

In a further preferred embodiment, a method according to the invention is characterized in that the composition (A) contains one or more organic C₁-C₆ alkoxy silanes (A1) of the formula (S-II)

(R₅O)_{c}(R₆)_{d}Si-(A)ₑ-[NR₇-(A′)]_{f}-[O-(A″)]_{g}-[NR₈-(A′″)]ₕ-Si(R₆′)_{d′}(OR₅′)_{c′} (S-II),

in which
e and f both represent the number 1,
g and h both represent the number 0,
A and A′ represent, independently of one another, a linear, divalent C₁-C₆ alkylene group,
   and
R₇ represents a hydrogen atom, a methyl group, a 2-hydroxyethyl group, a 2-alkenyl group, a 2-aminoethyl group or a grouping of formula (III).

In a further preferred embodiment, a method according to the invention is characterized in that the composition (A) contains one or more organic C₁-C₆ alkoxy silanes (A1) of formula (S-II), in which
e and f both represent the number 1,
g and h both represent the number 0,
A and A′ represent, independently of one another, a methylene group (—CH₂—), an ethylene group (—CH₂—CH₂—) or a propylene group (—CH₂—CH₂—CH₂), and
R₇ represents a hydrogen atom, a methyl group, a 2-hydroxyethyl group, a 2-alkenyl group, a 2-aminoethyl group or a grouping of formula (III).

Organic silicon compounds of the formula (S-II) which are well-suited for achieving the object according to the invention are

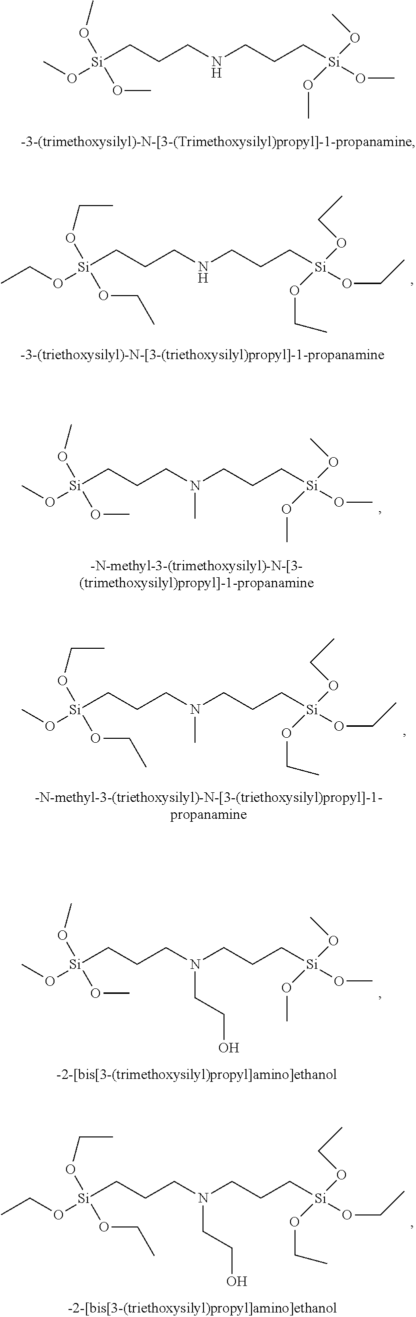

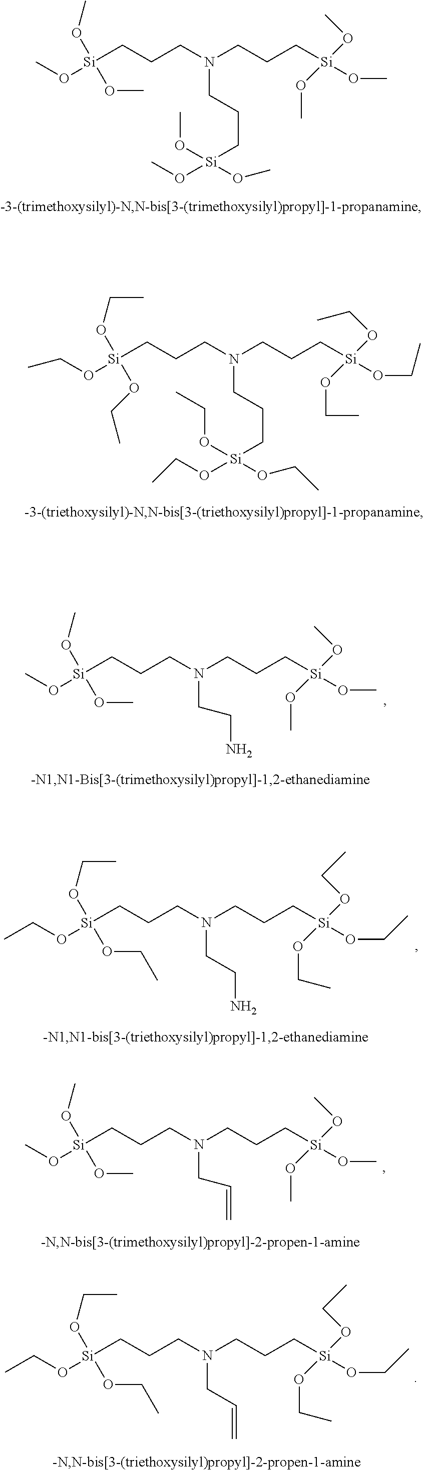

The aforementioned organic silicon compounds of the formula (S-II) are commercially available.

Bis(trimethoxysilylpropyl)amines having the CAS number 82985-35-1 can, for example, be purchased from Sigma-Aldrich.

Bis[3-(triethoxysilyl)propyl]amines having the CAS number 13497-18-2 can be purchased from Sigma-Aldrich, for example.

N-methyl-3-(trimethoxysilyl)-N-[3-(trimethoxysilyl)propyl]-1-propanamine is alternatively also referred to as bis(3-trimethoxysilylpropyl)-N-methylamine and is available commercially from Sigma-Aldrich or Fluorochem.

3-(triethoxysilyl)-N,N-bis[3-(triethoxysilyl)propyl]-1-propanamine having the CAS number 18784-74-2 can be purchased, for example, from Fluorochem or Sigma-Aldrich.

In a further preferred embodiment, a method according to the invention is characterized in that the composition (A) contains one or more organic C₁-C₆ alkoxy silanes of the formula (S-II) which are selected from the group consisting of
3-(trimethoxysilyl)-N-[3-(trimethoxysilyl)propyl]-1-propanamine,
3-(triethoxysilyl)-N-[3-(triethoxysilyl)propyl]-1-propanamine,
N-methyl-3-(trimethoxysilyl)-N-[3-(trimethoxysilyl)propyl]-1-propanamine,
N-methyl-3-(triethoxysilyl)-N-[3-(triethoxysilyl)propyl]-1-propanamine,
2-[bis[3-(trimethoxysilyl)propyl]amino]ethanol,
2-[bis[3-(triethoxysilyl)propyl]amino]ethanol,
3-(trimethoxysilyl)-N, N-bis[3-(trimethoxysilyl)propyl]-1-propanamine,
3-(triethoxysilyl)-N, N-bis[3-(triethoxysilyl)propyl]-1-propanamine,
N1, N1-bis[3-(trimethoxysilyl)propyl]-1,2-ethanediamine,
N1, N1-bis[3-(triethoxysilyl)propyl]-1,2-ethanediamine,
N,N-bis[3-(trimethoxysilyl)propyl]-2-propen-1-amine, and/or
N,N-bis[3-(triethoxysilyl)propyl]-2-propen-1-amine,
   and/or the condensation products thereof.

In further dyeing tests, it has likewise been found to be very particularly advantageous if at least one organic C₁-C₆ alkoxy silane (A1) of formula (S-IV) was used in the method according to the invention.

R₉Si(OR₁₀)ₖ(R₁₁)ₘ (S-IV).

The compounds of the formula (S-IV) are organic silicon compounds selected from silanes having one, two or three silicon atoms, the organic silicon compound comprising one or more hydrolyzable groups per molecule.

The organic silicon compound(s) of the formula (S-IV) can also be designated silanes of the type of the alkyl C₁-C₆ alkoxy-silanes,

R₉Si(OR₁₀)ₖ(R₁₁)ₘ (S-IV),

in which
R₉ represents a C₁-C₁₂ alkyl group,
R₁₀ represents a C₁-C₆ alkyl group,
R₁₁ represents a C₁-C₆ alkyl group,
k represents an integer from 1 to 3, and
m represents the integer 3-k.

In a further embodiment, a particularly preferred method according to the invention is characterized in that the first composition (A) contains one or more organic C₁-C₆ alkoxy silanes (A1) of formula (S-IV),

R₉Si(OR₁₀)ₖ(R₁₁)ₘ (S-IV),

in which
R₉ represents a C₁-C₁₂ alkyl group,
R₁₀ represents a C₁-C₆ alkyl group,
R₁₁ represents a C₁-C₆ alkyl group,
k represents an integer from 1 to 3, and
m represents the integer 3-k,
   and/or the condensation products thereof.

In the organic C₁-C₆ alkoxy silanes of the formula (S-IV), the group R₉ represents a C₁-C₁₂ alkyl group. This C₁-C₁₂ alkyl group is saturated and can be linear or branched. R₉ preferably represents a C₁-C₈ alkyl group. Preferably, R₉ represents a methyl group, an ethyl group, an n-propyl group, an n-butyl group, an n-pentyl group, an n-hexyl group, an n-octyl group or an n-dodecyl group.

Particularly preferably, R₉ represents a methyl group, an ethyl group or an n-octyl group.

In the organic silicon compounds of the formula (S-IV), the group R₁₀ represents a C₁-C₆ alkyl group. Particularly preferably, R₁₀ represents a methyl group or an ethyl group.

In the organic silicon compounds of the formula (S-IV), the group R₁₁ represents a C₁-C₆ alkyl group. Particularly preferably, R₁₁ represents a methyl group or an ethyl group.

Furthermore, k represents an integer from 1 to 3, and m represents the integer 3-k. If k represents the number 3, then m is equal to 0. If k represents the number 2, then m is equal to 1. If k represents the number 1, then m is equal to 2.

Dyeing having the best fastness to washing could be obtained when the composition (A) contains at least one organic C₁-C₆ alkoxy silane (A1) of the formula (S-IV), in which the group k represents the number 3. In this case, the group m represents the number 0.

Organic silicon compounds of the formula (S-IV) which are particularly well-suited for achieving the object according to the invention are

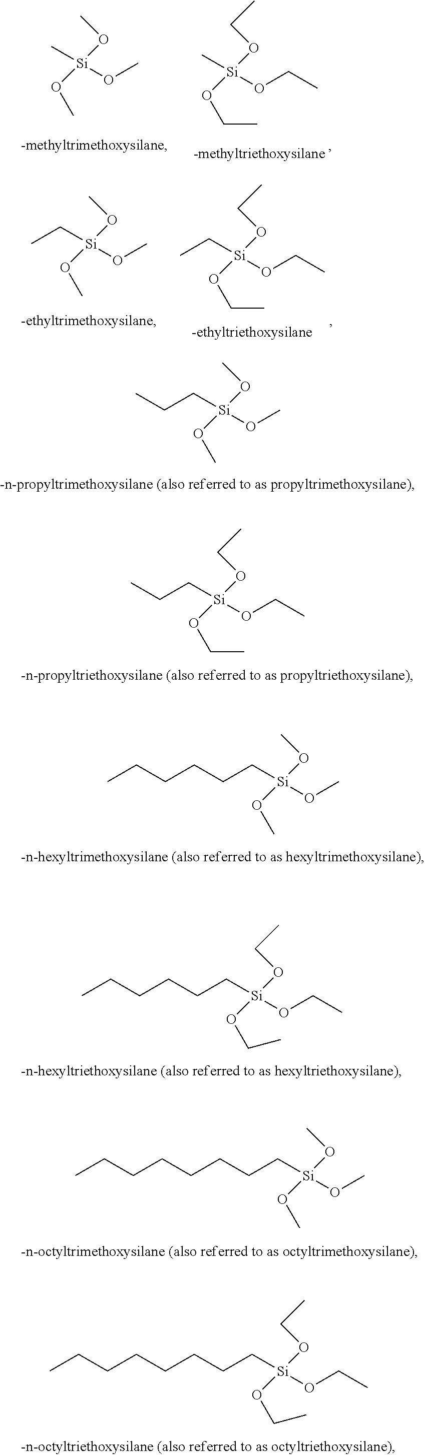

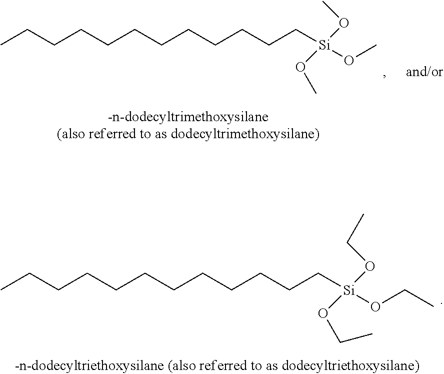

In a further preferred embodiment, a method according to the invention is characterized in that the first composition (A) contains at least one organic C₁-C₆ alkoxy silane (A1) of formula (S-IV), which is selected from the group consisting of
methyltrimethoxysilane,
methyltriethoxysilane,
ethyltrimethoxysilane,
ethyltriethoxysilane,
hexyltrimethoxysilane,
hexyltriethoxysilane,
octyltrimethoxysilane,
octyltriethoxysilane,
dodecyltrimethoxysilane,
dodecyltriethoxysilane,
   and/or the condensation products thereof.

Furthermore, it has proven to be very particularly preferred if the composition (A) contains both at least one organic C₁-C₆ alkoxy silane (A1) of the formula (S-I) and also at least one organic C₁-C₁₂ alkyl C₁-C₆ alkoxy silane (A2) of the formula (S-IV).

In a further preferred embodiment, a method according to the invention is characterized in that the composition (A) contains at least one organic C₁-C₆ alkoxy silane (A1) of the formula (S-I) and at least one organic C₁-C₆ alkoxy silane (A1) of the formula (S-IV).

Particularly preferably, the composition (A) contains the organic C₁-C₆ alkoxy silanes (A1) of the formula (S-I) and the organic C₁-C₁₂ alky C₁-C₆ alkoxy silanes (A2) of the formula (S-IV), in a specified quantity ratio.

In a further explicitly very particularly preferred embodiment, a method according to the invention is characterized in that the weight ratio of the total amount of the organic C₁-C₆ alkoxy silanes (A1) of the formula (S-I) contained in the composition (A) to the total amount of the organic C₁-C₁₂ alkyl C₁-C₆ alkoxy silanes of the formula (S-IV) contained in the composition (A), i.e. the weight ratio (Si-I)/(Si-IV), is at a value of from 0.1 to 5.0, preferably from 0.1 to 2.5, further preferably from 0.1 to 1.5, even more preferably from 0.1 to 1.0, and most preferably from 0.1 to 0.45.

The corresponding hydrolysis or condensation products are, for example, the following compounds. In this case, the condensation products represent maximum oligomeric compounds, but not polymers.

Hydrolysis of C₁-C₆ alkoxy silane of the formula (S-I) with water (reaction scheme using the example of 3-aminopropyltriethoxysilane):

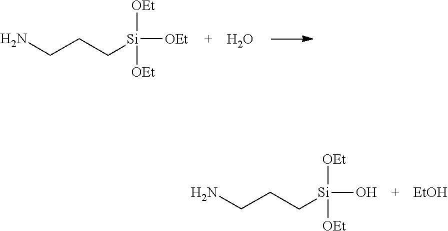 [SMILES: "EtOH" ; CCO[Si](CCCN)(OCC)OCC ; O ; CCO[Si](O)(CCCN)OCC]

Depending on the amount of water used, the hydrolysis reaction can also take place multiple times per C₁-C₆ alkoxy silane:

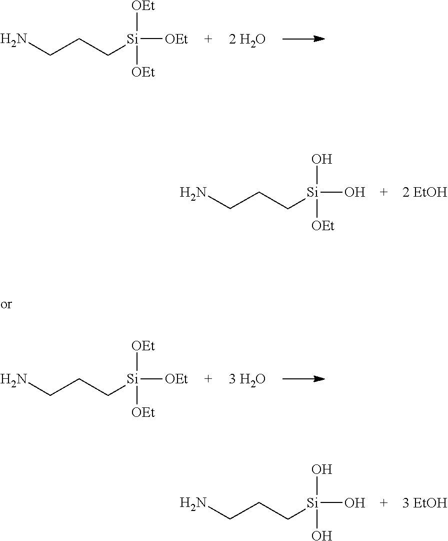

Hydrolysis of C₁-C₆ alkoxy silane of the formula (S-IV) with water (reaction scheme using the example of methyltrimethoxysilane):

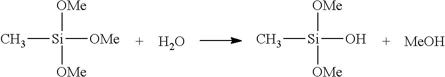

Depending on the amount of water used, the hydrolysis reaction can also take place multiple times per C₁-C₅ alkoxy silane:

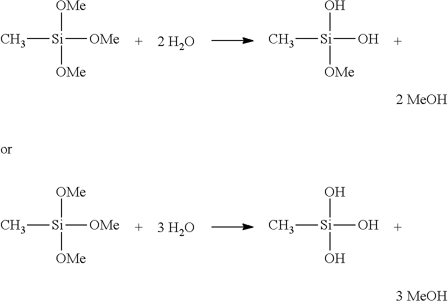

Possible condensation reactions are, for example (shown on the basis of the mixture (3-aminopropyl)triethoxysilane and methyltrimethoxysilane):

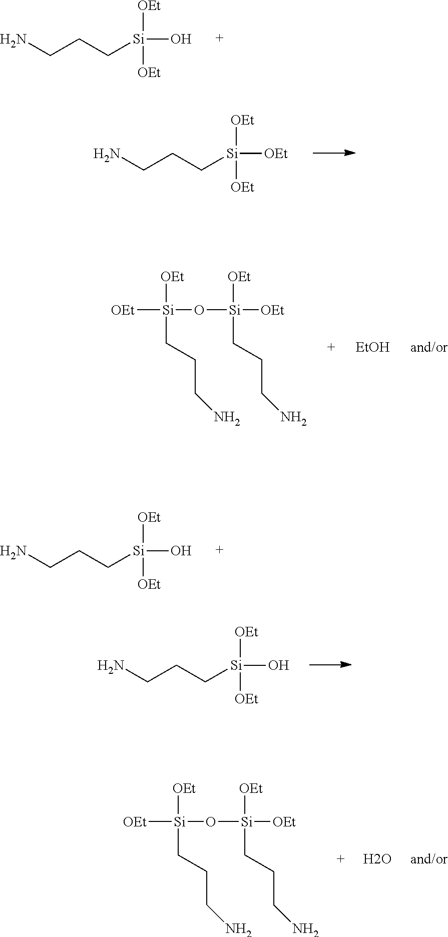

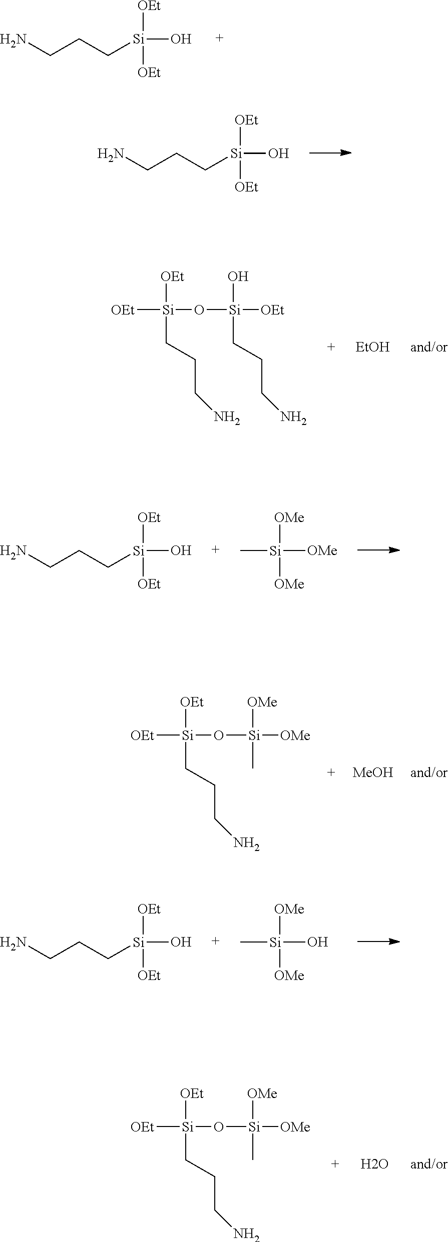

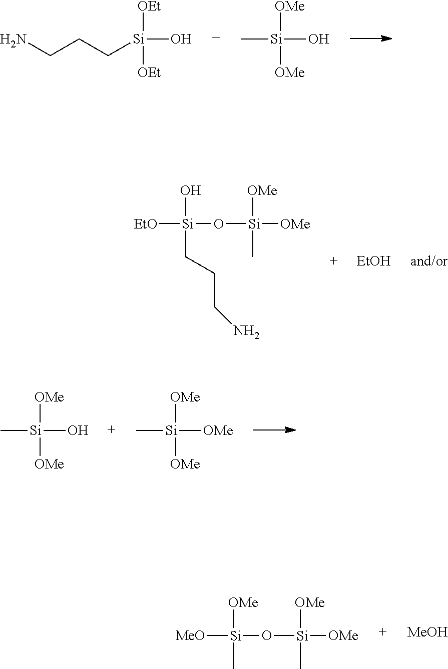

In the above reaction schemes, given by way of example, the condensation to form a dimer is shown in each case, but further condensations to oligomers having a plurality of silane atoms are also possible and preferred.

Both partially hydrolyzed and fully hydrolyzed C₁-C₆ alkoxy silanes of the formula (S-I), which undergo condensation with not yet reacted partially or else completely hydrolyzed C₁-C₆ alkoxy silanes of the formula (S-I), can be part of these condensation reactions. In this case, the C₁-C₆ alkoxy silanes of the formula (S-I) react with themselves.

Furthermore, both partially hydrolyzed and fully hydrolyzed C₁-C₆ alkoxy silanes of the formula (S-I), which undergo condensation with not yet reacted partially or else completely hydrolyzed C₁-C₆ alkoxy silanes of the formula (S-IV), can also be part of the condensation reactions. In this case, the C₁-C₆ alkoxy silanes of the formula (S-I) react with the C₁-C₆ alkoxy silanes of the formula (S-IV).

Furthermore, both partially hydrolyzed and fully hydrolyzed C₁-C₆ alkoxy silanes of the formula (S-IV), which undergo condensation with not yet reacted partially or else completely hydrolyzed C₁-C₆ alkoxy silanes of the formula (S-IV), can also be part of the condensation reactions. In this case, the C₁-C₆ alkoxy silanes of the formula (S-IV) react with themselves.

The composition (A) of the invention can contain one or more organic C₁-C₆ alkoxy silanes (A1) in different constituent amounts. This is determined by a person skilled in the art depending on the desired thickness of the silane coating on the keratin material, and on the amount of the keratin material to be treated.

Particularly storage-stable preparations having a very good dyeing result could be obtained, during use, when the composition (A)—based on the total weight of the composition (A)—contains one or more organic C₁-C₆ alkoxy silanes (A1) and/or the condensation products thereof in a total amount of 1.0 to 99.0 wt. %, preferably of 2.0 to 80.0 wt. %, more preferably of 3.0 to 60.0 wt. %, even more preferably of 4.0 to 40.0 wt. % and very particularly preferably of 5.0 to 15.0 wt. %.

In a further embodiment, a very particularly preferred method is characterized in that the composition (A)—based on the total weight of the composition (A)—contains one or more organic C₁-C₆ alkoxy silanes (A1) and/or the condensation products thereof in a total amount of 1.0 to 99.0 wt. %, preferably of 2.0 to 80.0 wt. %, more preferably of 3.0 to 60.0 wt. %, even more preferably of 4.0 to 40.0 wt. % and very particularly preferably of 5.0 to 15.0 wt. %.

### Further Cosmetic Ingredients in the Composition (A)

In addition, the composition (A) can also contain one or more further cosmetic ingredients.

The cosmetic ingredients which can optionally be used in the composition (A) can be all suitable components in order to give the agent further positive properties. For example, the composition (A) may contain a solvent, a surface-active compound from the group of non-ionic, cationic, anionic or zwitterionic/amphoteric surfactants, the dyeing compounds from the group of the pigments, the direct dyes, the oxidation dye precursors, the fatty components from the group of the C₈-C₃₀ fatty alcohols, the hydrocarbon compounds, fatty acid esters, the acids and bases belonging to the group of the pH regulators, perfumes, preservatives, plant extracts and protein hydrolyzates.

The selection of these additional substances is made by the skilled artisan according to the desired properties of the agents. With regard to other optional components and the amounts of said components used, reference is explicitly made to relevant handbooks known to a person skilled in the art.

### Water Content (A1) in the Composition (A)

The method according to the invention is characterized by the use of a composition (A) on the keratin material.

In order to ensure a sufficiently high storage stability, the composition (A) can be characterized in that it has a low water content, preferably is substantially anhydrous. Therefore, the composition (A) preferably contains, based on the total weight of the composition (A), less than 15 wt. % water.

At a water content of just below 15 wt. %, the compositions (A) are storage-stable over longer periods of time. However, in order to further improve the storage stability and to achieve a sufficiently high reactivity of the organic C₁-C₆ alkoxy silanes (A2), it has been found to be particularly preferred to yet further reduce the water content in the composition (A). For this reason, the composition (A) contains, based on the total weight of the composition (A), preferably 0.01 to 15.0 wt. %, preferably 0.1 to 13.0 wt. %, more preferably 0.5 to 11.0, and very preferably 1.0 to 9.0 wt. % water.

Within the context of a very particularly preferred embodiment, a method according to the invention is characterized in that the composition (A) contains—based on the total weight of the composition (A)—0.01 to 15.0 wt. %, preferably 0.1 to 13.0 wt. %, more preferably 0.5 to 11.0 wt. %, and very particularly preferably 1.0 to 9.0 wt. % water.

Within the context of a further embodiment, however, an aqueous composition (A) can also be applied to the keratin material. Within the context of this embodiment, a method according to the invention is characterized in that composition (A) contains, based on the total weight of the composition (A), 50.0 to 99.0 wt. %, preferably 60.0 to 98.0 wt. %, more preferably 65.0 to 97.0 wt. %, and particularly preferably 70.0 to 96.0 wt. % water.

### pH of the Compositions (A)

In further tests, it has been found that the pH values of the composition (A) can influence the color intensities obtained during the dyeing. It has been found here that in particular alkaline pH values have an advantageous effect on the dyeing performance that can be achieved in the method.

For this reason, it is preferred for the composition (A) to have a pH of from 7.0 to 12.0, preferably from 7.5 to 11.5, more preferably from 8.0 to 11.0, and very particularly preferably from 8.0 to 10.5.

The measurement of the pH can be carried out using the usual methods known from the prior art, such as the pH measurement by means of glass electrodes via combination electrodes or via pH indicator paper.

In a further very particularly preferred embodiment, a method according to the invention is characterized in that the composition (A) has a pH of 7.0 to 12.0, preferably 7.5 to 11.5, more preferably 8.0 to 11.0, and very particularly preferably 8.0 to 10.5.

### Dyeing Compounds in the Composition (B)

The method according to the invention comprises the application of a second composition (B) on the keratin material. In this case, the composition (B) is characterized in that it contains one or more dyeing compounds (B1) from the group of the pigments and the direct dyes.

Pigments within the meaning of the present invention are understood to mean dyeing compounds which have a solubility in water, at 25° C., of less than 0.5 g/L, preferably of less than 0.1 g/L, even more preferably of less than 0.05 g/L. The water solubility can be carried out, for example, by means of the method described hereinafter: 0.5 g of the pigment is weighed in a beaker. A stirring bar is added. Then, one liter of distilled water is added. This mixture is heated to 25° C., while stirring on a magnetic stirrer for one hour. If undissolved constituents of the pigment are still visible in the mixture after this period, then the solubility of the pigment is below 0.5 g/L. If the pigment-water mixture cannot be visually assessed due to the high intensity of the pigment that may be present finely dispersed, the mixture is filtered. If a proportion of undissolved pigments remains on the filter paper, the solubility of the pigment is below 0.5 g/L.

Suitable dye pigments may be of inorganic and/or organic origin.

In a preferred embodiment, the agent according to the invention is characterized in that it contains at least one dyeing compound from the group of the inorganic and/or organic pigments.

Preferred dye pigments are selected from synthetic or natural inorganic pigments. Inorganic dye pigments of natural origin can be produced, for example, from chalk, ocher, umbra, green earth, burnt sienna or graphite. Furthermore, black pigments, such as black iron oxide, colored pigments, such as ultramarine or red iron oxide, and also fluorescent or phosphorescent pigments, can be used as inorganic dye pigments.

Colored metal oxides, hydroxides and oxide hydrates, mixed phase pigments, sulfur-containing silicates, silicates, metal sulfides, complex metal cyanides, metal sulfates, chromates and/or molybdates are particularly suitable. Particularly preferred dye pigments are black iron oxide (CI 77499), yellow iron oxide (CI 77492), red and brown iron oxide (CI 77491), manganese violet (CI 77742), ultramarine (sodium aluminum sulphosilicates, CI 77007, Pigment Blue 29), chromium oxide hydrate (CI77289), iron blue (ferric ferrocyanide, CI77510) and/or carmine (cochineal).

Dyeing compounds from the group of the pigments which are also particularly preferred according to the invention are colored pearlescent pigments. These are usually based on mica and may be coated with one or more metal oxides. Mica is a phyllosilicate. The most important representatives of these silicates are muscovite, phlogopite, paragonite, biotite, lepidolite, and margarite. In order to prepare the pearlescent pigments in conjunction with metal oxides, the mica, primarily muscovite or phlogopite, is coated with a metal oxide.

Within the context of a further very particularly preferred embodiment, a method according to the invention is characterized in that the composition (B) contains at least one inorganic pigment, which is preferably selected from the group of the colored metal oxides, metal hydroxides, metal oxide hydrates, silicates, metal sulfides, complex metal cyanides, metal sulfates, bronze pigments and/or mica-based colored pigments which are coated with at least one metal oxide and/or a metal oxychloride.

As an alternative to natural mica, synthetic mica coated with one or more metal oxides(s) can optionally also be used as the pearlescent pigment. Particularly preferred pearlescent pigments are based on natural or synthetic mica and are coated with one or more of the aforementioned metal oxides. The color of the respective pigments can be varied by varying the layer thickness of the metal oxide(s).

In a further preferred embodiment, a method according to the invention is characterized in that the composition (B) contains at least one dyeing compound from the group of the pigments, selected from the group of colored metal oxides, metal hydroxides, metal oxide hydrates, silicates, metal sulfides, complex metal cyanides, metal sulphates, bronze pigments and/or from mica-based dyeing compounds which are coated with at least one metal oxide and/or metal oxychloride.

In a further preferred embodiment, a method according to the invention is characterized in that the composition (B) contains at least one dyeing compound which is selected from mica-based pigments which are coated with one or more metal oxides from the group of titanium dioxide (CI 77891), black iron oxide (CI 77499), yellow iron oxide (CI 77492), red and/or brown iron oxide (CI 77491, CI 77499), manganese violet (CI 77742), ultramarine (sodium aluminum sulfosilicates, CI 77007, Pigment Blue 29), chromium oxide hydrate (CI 77289), chromium oxide (CI 77288) and/or iron blue (ferric ferrocyanide, CI 77510).

Examples of particularly suitable dye pigments are commercially available for example under the trade names Rona®, Colorona®, Xirona®, Dichrona® and Tiimron® from the company Merck, Ariabel® and Unipure® from the company Sensient, Prestige® from the company Eckart, Cosmetic Colors and Sunshine® from the company Sunstar.

Very particularly preferred dye pigments having the trade name Colorona® are, for example:
Colorona Copper, Merck, MICA, CI 77491 (IRON OXIDES)
Colorona Passion Orange, Merck, Mica, CI 77491 (Iron Oxides), Alumina
Colorona Patina Silver, Merck, MICA, CI 77499 (IRON OXIDES), CI 77891 (TITANIUM DIOXIDE)
Colorona RY, Merck, CI 77891 (TITANIUM DIOXIDE), MICA, CI 75470 (CARMINE)
Colorona Oriental Beige, Merck, MICA, CI 77891 (TITANIUM DIOXIDE), CI 77491 (IRON OXIDES)
Colorona Dark Blue, Merck, MICA, TITANIUM DIOXIDE, FERRIC FERROCYANIDE
Colorona Chameleon, Merck, CI 77491 (IRON OXIDES), MICA
Colorona Aborigine Amber, Merck, MICA, CI 77499 (IRON OXIDES), CI 77891 (TITANIUM DIOXIDE)
Colorona Blackstar Blue, Merck, CI 77499 (IRON OXIDES), MICA
Colorona Patagonian Purple, Merck, MICA, CI 77491 (IRON OXIDES), CI 77891 (TITANIUM DIOXIDE), CI 77510 (FERRIC FERROCYANIDE)
Colorona Red Brown, Merck, MICA, CI 77491 (IRON OXIDES), CI 77891 (TITANIUM DIOXIDE)
Colorona Russet, Merck, CI 77491 (TITANIUM DIOXIDE), MICA, CI 77891 (IRON OXIDES)
Colorona Imperial Red, Merck, MICA, TITANIUM DIOXIDE (CI 77891), D&C RED NO. 30 (CI 73360)
Colorona Majestic Green, Merck, CI 77891 (TITANIUM DIOXIDE), MICA, CI 77288 (CHROMIUM OXIDE GREENS)
Colorona Light Blue, Merck, MICA, TITANIUM DIOXIDE (CI 77891), FERRIC FERROCYANIDE (CI 77510)
Colorona Red Gold, Merck, MICA, CI 77891 (TITANIUM DIOXIDE), CI 77491 (IRON OXIDES)
Colorona Gold Plus MP 25, Merck, MICA, TITANIUM DIOXIDE (CI 77891), IRON OXIDES (CI 77491)
Colorona Carmine Red, Merck, MICA, TITANIUM DIOXIDE, CARMINE
Colorona Blackstar Green, Merck, MICA, CI 77499 (IRON OXIDES)
Colorona Bordeaux, Merck, MICA, CI 77491 (IRON OXIDES)
Colorona Bronze, Merck, MICA, CI 77491 (IRON OXIDES)
Colorona Bronze Fine, Merck, MICA, CI 77491 (IRON OXIDES)
Colorona Fine Gold MP 20, Merck, MICA, CI 77891 (TITANIUM DIOXIDE), CI 77491 (IRON OXIDES)
Colorona Sienna Fine, Merck, CI 77491 (IRON OXIDES), MICA
Colorona Sienna, Merck, MICA, CI 77491 (IRON OXIDES)
Colorona Precious Gold, Merck, Mica, CI 77891 (Titanium dioxide), Silica, CI 77491(Iron oxides), Tin oxide
Colorona Sun Gold Sparkle MP 29, Merck, MICA, TITANIUM DIOXIDE, IRON OXIDES, MICA, CI 77891, CI 77491 (EU)
Colorona Mica Black, Merck, CI 77499 (Iron oxides), Mica, CI 77891 (Titanium dioxide)
Colorona Bright Gold, Merck, Mica, CI 77891 (Titanium dioxide), CI 77491(Iron oxides)
Colorona Blackstar Gold, Merck, MICA, CI 77499 (IRON OXIDES)

Further particularly preferred dye pigments having the trade name Xirona® are, for example:
Xirona Golden Sky, Merck, Silica, CI 77891 (Titanium Dioxide), Tin Oxide
Xirona Caribbean Blue, Merck, Mica, CI 77891 (Titanium Dioxide), Silica, Tin Oxide
Xirona Kiwi Rose, Merck, Silica, CI 77891 (Titanium Dioxide), Tin Oxide
Xirona Magic Mauve, Merck, Silica, CI 77891 (Titanium Dioxide), Tin Oxide.

In addition, particularly preferred dye pigments having the trade name Unipure® are, for example:
Unipure Red LC 381 EM, Sensient CI 77491 (Iron Oxides), Silica
Unipure Black LC 989 EM, Sensient, CI 77499 (Iron Oxides), Silica
Unipure Yellow LC 182 EM, Sensient, CI 77492 (Iron Oxides), Silica
Timiron Synwhite Satin, Merck, Synthetic Fluorphlogopite, Titanium Dioxide, Tin Oxide
Timiron Super Blue, Merck, Mica, CI 77891 (Titanium Dioxide)
Timiron Diamond Cluster MP 149, Merck, Mica, CI 77891 (Titanium dioxide)
Timiron Splendid Gold, Merck, CI 77891 (Titan dioxide), Mica, Silica
Timiron Super Sulver, Merck, Mica, CI 77891 (Titan dioxide)

Within the context of a further embodiment, the composition (B) can also contain one or more dyeing compounds from the group of the organic pigments.

The organic pigments according to the invention are correspondingly insoluble, organic dyes or color varnishes, which may be selected, for example, from the group of nitroso, nitro, azo, xanthene, anthraquinone, isoindolinone, isoindoline, quinacridone, perinone, perylene, diketopyrrolopyrrole, indigo, thioindido, dioxazine, and/or triarylmethane compounds.

For example carmine, quinacridone, phthalocyanine, sorghum, blue pigments having the Color Index Numbers CI 42090, CI 69800, CI 69825, CI 73000, CI 74100, CI 74160, yellow pigments having the Color Index Numbers CI 11680, CI 11710, CI 15985, CI 19140, CI 20040, CI 21100, CI 21108, CI 47000, CI 47005, green pigments having the Color Index Numbers CI 61565, CI 61570, CI 74260, orange pigments having the Color Index Numbers CI 11725, CI 15510, CI 45370, CI 71105, red pigments having the Color Index Numbers CI 12085, CI 12120, CI 12370, CI 12420, CI 12490, CI 14700, CI 15525, CI 15580, CI 15620, CI 15630, CI 15800, CI 15850, CI 15865, CI 15880, CI 17200, CI 26100, CI 45380, CI 45410, CI 58000, CI 73360, CI 73915 and/or CI 75470 can be cited as particularly well-suited organic pigments.

In a further particularly preferred embodiment, a method according to the invention is characterized in that the composition (B) contains at least one organic pigment which is preferably selected from the group of carmine, quinacridone, phthalocyanine, sorghum, blue pigments having the Color Index Numbers CI 42090, CI 69800, CI 69825, CI 73000, CI 74100, CI 74160, yellow pigments having the Color Index Numbers CI 11680, CI 11710, CI 15985, CI 19140, CI 20040, CI 21100, CI 21108, CI 47000, CI 47005, green pigments having the Color Index Numbers CI 61565, CI 61570, CI 74260, orange pigments having the Color Index Numbers CI 11725, CI 15510, CI 45370, CI 71105, red pigments having the Color Index Numbers CI 12085, CI 12120, CI 12370, CI 12420, CI 12490, CI 14700, CI 15525, CI 15580, CI 15620, CI 15630, CI 15800, CI 15850, CI 15865, CI 15880, CI 17200, CI 26100, CI 45380, CI 45410, CI 58000, CI 73360, CI 73915 and/or CI 75470.

The organic pigment can furthermore also be a color varnish. The term color varnish is understood, within the meaning of the invention, to mean particles which comprise a layer of absorbed dyes, the unit consisting of particle and dye being insoluble under the above-mentioned conditions. The particles may be, for example, inorganic substrates, which may be aluminum, silica, calcium borosilicate, calcium aluminum borosilicate or aluminum.

For example, the Alizarin color varnish can be used as color varnish.

Due to their excellent light and temperature resistance, the use of the aforementioned pigments in the agent according to the invention is particularly preferred. It is furthermore preferred if the pigments used have a specific particle size. This particle size on the one hand leads to a uniform distribution of the pigments in the polymer film formed and, on the other hand, avoids a rough hair or skin feel after the application of the cosmetic agent. It is therefore advantageous according to the invention if the at least one pigment has an average particle size D₅₀ from 1.0 to 50 μm, preferably from 5.0 to 45 μm, preferably from 10 to 40 μm, in particular from 14 to 30 μm. The average particle size D₅₀ can be determined, for example, using dynamic light scattering (DLS).

For the dyeing of the keratin material, pigments of a specific shape may also have been used. For example, a pigment based on a lamellar and/or lenticular small substrate plate may be used. Furthermore, the dyeing is also possible based on a small substrate plate which comprises a vacuum metalized pigment.

Within the context of a further embodiment, the composition (A) and/or the composition (B) and/or an optionally usable composition (C) can also comprise one or more dyeing compounds from the group of the pigments based on a lamellar small substrate plate, the pigments based on a lenticular small substrate plate, and the vacuum metalized pigments.

The small substrate plates of this type have an average thickness of at most 50 nm, preferably less than 30 nm, particularly preferably at most 25 nm, for example at most 20 nm. The average thickness of the small substrate plates is at least 1 nm, preferably at least 2.5 nm, particularly preferably at least 5 nm, for example at least 10 nm. Preferred ranges for the thickness of the small substrate plates are 2.5 to 50 nm, 5 to 50 nm, 10 to 50 nm; 2.5 to 30 nm, 5 to 30 nm, 10 to 30 nm; 2.5 to 25 nm, 5 to 25 nm, 10 to 25 nm, 2.5 to 20 nm, 5 to 20 nm and 10 to 20 nm. Preferably, each small substrate plate has as uniform a thickness as possible.

Due to the small thickness of the small substrate plates, the pigment has a particularly high covering capacity.

The small substrate plates are constructed monolithically. Monolithic means, in this context, consisting of a single closed unit without fractures, layers or inclusions, it being possible, however, for structural changes to occur within the small substrate plates. The small substrate plates are preferably constructed homogeneously, i.e. no concentration gradient occurs within the small plates. In particular, the small substrate plates are not constructed in layers and have no particles distributed therein.

The size of the small substrate plate can be matched to the respective application, in particular to the desired effect on the keratin material. In general, the small substrate plates have an average largest diameter of approximately 2 to 200 μm, in particular approximately 5 to 100 μm.

In a preferred embodiment, the form factor (aspect ratio), expressed by the ratio of the average size to the average thickness, is at least 80, preferably at least 200, more preferably at least 500, particularly preferably more than 750. In this case, the average size of the uncoated small substrate plates is understood to mean the d50 value of the uncoated small substrate plates. Unless stated otherwise, the d50 value was determined using a device of the Sympatec Helos type, having QUIXEL wet dispersion. In this case, for sample preparation, the sample to be investigated was pre-dispersed in isopropanol for a period of 3 minutes.

The small substrate plates may be constructed from any material that can be made into the form of a small plate.

They can be of natural origin, but can also be produced synthetically. Materials from which the small substrate plates can be constructed are, for example, metals and metal alloys, metal oxides, preferably aluminum oxide, inorganic compounds, and minerals such as mica and (semi-) precious stones, as well as plastics materials. Preferably, the small substrate plate are made of metal (alloy)s.

Any metal suitable for metallic pearlescent pigments is possible as the metal. Such metals are, inter alia, iron and steel, and all air-resistant and water-resistant (semi-) metals, such as platinum, zinc, chromium, molybdenum and silicon, as well as the alloys thereof, such as aluminum bronzes and brass. Preferred metals are aluminum, copper, silver and gold. Preferred small substrate plates are small aluminum plates and small brass plates, small substrate plates made of aluminum being particularly preferred.

Lamellar small substrate plates are characterized by an irregularly structured edge, and are also referred to as “cornflakes” due to their appearance.

Due to their irregular structure, pigments based on lamellar small substrate plates produce a high fraction of scattered light. In addition, the pigments based on lamellar small substrate plates do not completely cover the existing color of a keratin material and, for example, effects can be achieved analogously to a natural graying.

Lenticular (=lens-shaped) small substrate plates have a substantially regular round edge and are also referred to as “silver dollars” due to their appearance. Due to their regular structure, the fraction of the reflected light predominates in the case of pigments based on lenticular small substrate plates.

Vacuum metalized pigments (VMP) can be obtained, for example, by releasing metals, metal alloys or metal oxides from correspondingly coated films. These are characterized by a particularly small thickness of the small substrate plates in the range from 5 to 50 nm and by a particularly smooth surface having increased reflectivity. Small substrate plates which comprise a pigment metalized in vacuo are also referred to, within the context of this application, as VMP small substrate plates. VMP small substrate plates made of aluminum can be obtained, for example, by releasing aluminum from metalized films.

The small substrate plates made of metal or metal alloy can be passivated, for example by anodizing (oxide layer) or chromatizing.

Uncoated lamellar, lenticular and/or VPM small substrate plates, in particular those made of metal or metal alloy, reflect the incident light to a high degree and produce a light-dark flop, but no color impression.

A color impression can be created, for example, due to optical interference effects. Pigments of this kind can be based on at least single-coated small substrate plates. These demonstrate interference effects by superimposition of differently refracted and reflected light beams.

Accordingly, preferred pigments are pigments based on a coated small lamellar substrate plate. The small substrate plate preferably has at least one coating B of a highly refractive metal oxide having a coating thickness of at least 50 nm. A coating A is preferably also located between the coating B and the surface of the small substrate plate. Optionally, a further coating C is located on the layer B, which coating is different from the underlying layer B.

All substances which can be applied to the small substrate plates in a film-like and permanent manner and, in the case of layers A and B, have the required optical properties, are suitable as materials for the coatings A, B and C. In general, a coating of a part of the surface of the small substrate plates is sufficient to obtain a pigment having a glossy effect. Thus, for example, only the upper and/or lower side of the small substrate plates can be coated, the side face(s) being omitted. Preferably, the entire surface of the optionally passivated small substrate plates, including the side surfaces, is covered by coating B. The small substrate plates are thus completely enveloped by coating B. This improves the optical properties of the pigment and increases the mechanical and chemical resilience of the pigments. The above also applies for the layer A and preferably also for the layer C, if present.

Although a plurality of coatings A, B and/or C can be present in each case, the coated small substrate plates preferably each have only one coating A, B and, if present, C.

The coating B is constructed from at least one highly refractive metal oxide. Highly refractive materials have a refractive index of at least 1.9, preferably at least 2.0, particularly preferably at least 2.4. The coating B preferably comprises at least 95 wt. %, particularly preferably at least 99 wt. %, highly refractive metal oxide(s).

The coating B has a thickness of at least 50 nm. The thickness of coating B is preferably no more than 400 nm, particularly preferably no more than 300 nm.

Highly refractive metal oxides suitable for coating B are preferably selectively light-absorbing (i.e. colored) metal oxides such as iron(III) oxide (α- and γ-Fe2O3, red), cobalt(II) oxide (blue), chromium(III) oxide (green), titanium(III) oxide (blue, usually in a mixture with titanium oxynitrides and titanium nitrides) and vanadium(V) oxide (orange), and the mixtures thereof. Colorless highly refractive oxides, such as titanium dioxide and/or zirconium oxide are also suitable.

Coating B can contain a selectively absorbing dye, preferably 0.001 to 5 wt. %, particularly preferably 0.01 to 1 wt. %, in each case based on the total amount of the coating B. Organic and inorganic dyes which can be incorporated stably into a metal oxide coating are suitable.

The coating A preferably has at least one low-refractive metal oxide and/or metal oxide hydrate. Preferably, coating A comprises at least 95 wt. %, particularly preferably at least 99 wt. %, of low-refractive metal oxide (hydrate). Low-refractive materials have a refractive index of at most 1.8, preferably at most 1.6.

The low-refractive metal oxides suitable for coating A include, for example, silicon (di)oxide, silicon oxide hydrate, aluminum oxide, aluminum oxide hydrate, boron oxide, germanium oxide, manganese oxide, magnesium oxide and mixtures thereof, silicon dioxide being preferred. The coating A preferably has a thickness of 1 to 100 nm, particularly preferably 5 to 50 nm, particularly preferably 5 to 20 nm.

The distance between the surface of the small substrate plates and the inner surface of coating B is preferably at most 100 nm, more preferably at most 50 nm, particularly preferably at most 20 nm. Since the thickness of coating A, and thus the distance between the surface of the small substrate plates and coating B, is in the range indicated above, it can be ensured that the pigments have a high covering capacity.

If the pigment based on a lamellar small substrate plate has only one layer A, it is preferred for the pigment to have a lamellar small substrate plate made of aluminum and a layer A of silicon dioxide. If the pigment based on a lamellar small substrate plate has a layer A and a layer B, it is preferred for the pigment to comprise a lamellar small substrate plate made of aluminum, a layer A of silicon dioxide, and a layer B of iron oxide.

According to a preferred embodiment, the pigments have a further coating C of a metal oxide (hydrate) which is different from the underlying coating B. Suitable metal oxides are, for example, silicon (di)oxide, silicon oxide hydrate, aluminum oxide, aluminum oxide hydrate, zinc oxide, tin oxide, titanium dioxide, zirconium oxide, iron(III)oxide and chromium(III)oxide. Silicon dioxide is preferred.

The coating C preferably has a thickness of 10 to 500 nm, particularly preferably 50 to 300 nm. By providing the coating C, for example based on TiO2, a better interference can be achieved, a high covering capacity still being ensured.

Layers A and C serve in particular as corrosion protection and also for chemical and physical stabilization. The layers A and C particularly preferably contain silicon dioxide or aluminum oxide which are applied by the sol-gel process. This process comprises dispersing the uncoated lamellar small substrate plates or the lamellar small substrate plates already coated with layer A and/or layer B in a solution of a metal alkoxide, such as tetraethyl orthosilicate or aluminum triisopropanolate (usually in a solution of organic solvent or a mixture of organic solvent and water with at least 50 wt. % organic solvent, such as a C1 to C4-alcohol), and addition of a weak base or acid for hydrolyzing the metal alkoxide, thereby forming a film of the metal oxide on the surface of the (coated) small substrate plates.

The layer B can be produced, for example, by hydrolytic decomposition of one or more organic metal compounds and/or by precipitation of one or more dissolved metal salts, and an optional subsequent post-treatment (for example transfer of a formed hydroxide-containing layer into the oxide layers by tempering).

Although each of the coatings A, B and/or C can be constructed from a mixture of two or more metal oxide (hydrate)s, each of the coatings is preferably constructed from one metal oxide (hydrate).

The pigments based on coated lamellar or lenticular small substrate plates or the pigments based on coated VMP small substrate plates preferably have a thickness of 70 to 500 nm, particularly preferably 100 to 400 nm, especially preferably 150 to 320 nm, for example 180 to 290 nm. Due to the small thickness of the small substrate plates, the pigment has a particularly high covering capacity. The small thickness of the coated small substrate plates is achieved in particular by the fact that the thickness of the uncoated small substrate plates is low, but also in that the thicknesses of the coatings A and, if present, C, are set to the smallest possible value. The thickness of coating B determines the color impression of the pigment.

The adhesion and abrasion resistance of pigments based on coated small substrate plates in the keratin material can be significantly increased in that the outermost layer, depending on the structure layer A, B or C, is additionally modified by an organic compound, such as silanes, phosphoric esters, titanates, borates or carboxylic acids. In this case, the organic compounds are bound to the surface of the outermost, preferably metal oxide-containing, layer A, B or C. The outermost layer denotes the layer which is spatially furthest removed from the lamellar small substrate plate. The organic compounds are preferably functional silane compounds which can bind to the metal oxide-containing layer A, B or C. These may be both mono- and bifunctional compounds. Examples of bifunctional organic compounds are methacryloxypropenyltrimethoxysilane, 3-methacryloyloxypropyltrimethoxysilane, 3-acrylyloxypropyltrimethoxysilane, 2-acryloyloxyethyltrimethoxysilane, 3-methacryloyloxypropyltriethoxysilane, 3-acryloyloxyethyltriethoxysilane, 3-methacryloyloxypropyltris(methoxyethoxy) silane, 3-methacryloyloxypropyltris(butoxyethoxy) silane, 3-methacryloyloxypropyltris (butoxyethoxy) silane, 3-methacryloxypropyltris (propoxy) silane, 3-methacryloyloxypropyltris (butoxy) silane, 3-acryloxypropyltris (methoxyethoxy) silane, 3-acryloxypropyltris (butoxyethoxy) silane, 3-acryloyloxypropyltris (butoxy) silane, vinyltrimethoxysilane, vinyltriethoxysilane, vinylethyldichlorosilane, vinylmethyldiethoxysilane, vinylmethyldichlorosilane, vinylmethyldiethoxysilane, or phenylallyldichlorosilane. Furthermore, a modification with a monofunctional silane, in particular an alkylsilane or arylsilane, can take place. This has only one functional group, which can bind covalently to the surface of the pigment based on coated small lamellar substrate plates (i.e. to the outermost metal oxide-containing layer) or, when the covering is not entirely complete, to the metal surface. The hydrocarbyl of the silane faces away from the pigment. Depending on the nature and state of the hydrocarbyl of the silane, a different degree of hydrophobization of the pigment is achieved. Examples of such silanes are hexadecyltrimethoxysilane, propyltrimethoxysilane, etc. Pigments based on silicon dioxide-coated aluminum small substrate plates surface-modified with a monofunctional silane are particularly preferred. Octyltrimethoxysilane, octyltriethoxysilane, heptadecyl-trimethoxysilane and hecadecyltriethoxysilane are particularly preferred. As a result of the altered surface properties/hydrophobization, an improvement with regard to adhesion, abrasion resistance and orientation in the application can be achieved.

Suitable pigments based on a lamellar small substrate plate include, for example, the pigments of the VISIONAIRE series by Eckart.

Pigments based on a lenticular small substrate plate are available, for example, under the name Alegrace® Gorgeous from the company Schlenk Metallic Pigments GmbH.

Pigments based on a small substrate plate, which comprises a vacuum metalized pigment, are available, for example, under the name Alegrace® Marvelous or Alegrace® Aurous from the company Schlenk Metallic Pigments GmbH.

Within the context of a further embodiment, a method according to the invention is characterized in that the composition (A) contains, based on the total weight of the composition (A), one or more pigments in a total amount of from 0.001 to 20 wt. %, in particular from 0.05 to 5 wt. %.

Within the context of a further embodiment, a method according to the invention is characterized in that the composition (B) contains, based on the total weight of the composition (B), one or more pigments in a total amount of from 0.001 to 20 wt. %, in particular from 0.05 to 5 wt. %.

The compositions according to the invention can also contain one or more direct dyes as dyeing compounds. The direct dyes are dyes which attach directly to the hair and require no oxidative process to form the color. Direct dyes are usually nitrophenylenediamines, nitroaminophenols, azo dyes, anthraquinones, triarylmethane dyes, or indophenols.

The direct dyes within the meaning of the present invention have a solubility in water (760 mmHg) at 25° C. of more than 0.5 g/L and are therefore not to be regarded as pigments. Within the meaning of the present invention, the direct dyes preferably have a solubility in water (760 mmHg) at 25° C. of more than 1.0 g/L. Within the meaning of the present invention, the direct dyes particularly preferably have a solubility in water (760 mmHg) at 25° C. of more than 1.5 g/L.

Direct dyes can be divided into anionic, cationic, and non-ionic direct dyes.

In a further preferred embodiment, an agent according to the invention is characterized in that it contains at least one anionic, cationic and/or non-ionic direct dye as a dyeing compound.

In a further preferred embodiment, a method according to the invention is characterized in that the composition (B) and/or the composition (C) contains at least one dyeing compound from the group of the anionic, non-ionic, and/or cationic direct dyes.

Suitable cationic direct dyes are for example Basic Blue 7, Basic Blue 26, Basic Violet 2 and Basic Violet 14, Basic Yellow 57, Basic Red 76, Basic Blue 16, Basic Blue 347 (Cationic Blue 347 / Dystar), HC Blue No. 16, Basic Blue 99, Basic Brown 16, Basic Brown 17, Basic Yellow 57, Basic Yellow 87, Basic Orange 31, Basic Red 51 Basic Red 76.

In particular, non-ionic nitro dyes and quinone dyes and neutral azo dyes, for example, can be used as non-ionic direct dyes. Suitable non-ionic direct dyes are the compounds known under the international designations or trade names HC Yellow 2, HC Yellow 4, HC Yellow 5, HC Yellow 6, HC Yellow 12, HC Orange 1, Disperse Orange 3, HC Red 1, HC Red 3, HC Red 10, HC Red 11, HC Red 13, HC Red BN, HC Blue 2, HC Blue 11, HC Blue 12, Disperse Blue 3, HC Violet 1, Disperse Violet 1, Disperse Violet 4, Disperse Black 9, and 1,4-diamino-2-nitrobenzene, 2-amino-4-nitrophenol, 1,4-bis-(2-hydroxyethyl)amino-2-nitrobenzene, 3-nitro-4-(2-hydroxyethyl)aminophenol, 2-(2-hydroxyethyl)amino-4,6-dinitrophenol, 4-[(2-hydroxyethyl)amino]-3-nitro-1-methylbenzene, 1-amino-4-(2-hydroxyethyl)amino-5-chloro-2-nitrobenzene, 4-amino-3-nitrophenol, 1-(2′-ureidoethyl)amino-4-nitrobenzene, 2-[(4-amino-2-nitrophenyl)amino]benzoic acid, 6-nitro-1,2,3,4-tetrahydroquinoxaline, 2-hydroxy-1,4-naphthoquinone, picramic acid and the salts thereof, 2-amino-6-chloro-4-nitrophenol, 4-ethylamino-3-nitrobenzoic acid, and 2-chloro-6-ethylamino-4-nitrophenol.

Anionic direct dyes are also referred to as acid dyes. Acid dyes are understood to be direct dyes that have at least one carboxylic acid grouping (—COOH) and/or a sulfonic acid grouping (—SO₃H). Depending on the pH, the protonated forms (—COOH, —SO₃H) of carboxylic acid or sulfonic acid groupings are present in equilibrium with their deprotonated forms (—COO⁻, —SO₃⁻). The proportion of the protonated forms increases with a decreasing pH. If direct dyes are used in the form of their salts, the carboxylic acid groups or sulfonic acid groups are then present in deprotonated form and are neutralized, to maintain the electroneutrality, with corresponding stoichiometric equivalents of cations. Acid dyes according to the invention can also be used in the form of their sodium salts and/or their potassium salts.

The acid dyes within the meaning of the present invention have a solubility in water (760 mmHg) at 25° C. of more than 0.5 g/L and are therefore not to be regarded as pigments. Within the meaning of the present invention, the acid dyes preferably have a solubility in water (760 mmHg) at 25° C. of more than 1.0 g/L.

The alkaline earth salts (for example calcium salts and magnesium salts) or aluminum salts of acid dyes often have poorer solubility than the corresponding alkali salts. If the solubility of these salts is below 0.5 g/L (25° C., 760 mmHg), they do not fall under the definition of a direct dye.

An essential feature of the acid dyes is their ability to form anionic charges, the carboxylic acid groups or sulfonic acid groups responsible for usually being linked to different chromophoric systems. Suitable chromophoric systems are found, for example, in the structures of nitrophenylenediamines, nitroaminophenols, azo dyes, anthraquinone dyes, triarylmethane dyes, xanthene dyes, rhodamine dyes, oxazine dyes and/or indophenol dyes.

For example, one or more compounds from the following group can be selected as particularly suitable acid dyes: Acid Yellow 1 (D&C Yellow 7, Citronin A, Ext. D&C Yellow No. 7, Japan Yellow 403, CI 10316, COLIPA n° B001), Acid Yellow 3 (COLIPA n°: C 54, D&C Yellow no. 10, Quinoline Yellow, E104, Food Yellow 13), Acid Yellow 9 (CI 13015), Acid Yellow 17 (CI 18965), Acid Yellow 23 (COLIPA no. C 29, Covacap Jaune W 1100 (LCW), Sicovit Tartrazine 85 E 102 (BASF), Tartrazine, Food Yellow 4, Japan Yellow 4, FD&C Yellow no. 5), Acid Yellow 36 (CI 13065), Acid Yellow 121 (CI 18690), Acid Orange 6 (CI 14270), Acid Orange 7 (2-naphthol orange, Orange II, CI 15510, D&C Orange 4, COLIPA no. C015), Acid Orange 10 (C.I. 16230; Orange G sodium salt), Acid Orange 11 (CI 45370), Acid Orange 15 (CI 50120), Acid Orange 20 (CI 14600), Acid Orange 24 (BROWN 1;CI 20170;KATSU201;nosodiumsalt;Brown No.201;RESORCIN BROWN; ACID ORANGE 24; Japan Brown 201;D & C Brown no. 1), Acid Red 14 (C.I. 14720), Acid Red 18 (E124, Red 18; CI 16255), Acid Red 27 (E 123, CI 16185, C-Rot 46, Echtrot D, FD&C Red No.2, Food Red 9, Naphtholrot S), Acid Red 33 (Red 33, Fuchsia Red, D&C Red 33, CI 17200), Acid Red 35 (CI C.I. 18065), Acid Red 51 (CI 45430, Pyrosin B, Tetraiodfluorescein, Eosin J, lodeosin), Acid Red 52 (CI 45100, Food Red 106, Solar Rhodamine B, Acid Rhodamine B, Red no. 106 Pontacyl Brilliant Pink), Acid Red 73 (CI CI 27290), Acid Red 87 (Eosin, CI 45380), Acid Red 92 (COLIPA no. C53, CI 45410), Acid Red 95 (CI 45425, Erythtosine, Simacid Erythrosine Y), Acid Red 184 (CI 15685), Acid Red 195, Acid Violet 43 (Jarocol Violet 43, Ext. D&C Violet no. 2, CI 60730, COLIPA n° C063), Acid Violet 49 (CI 42640), Acid Violet 50 (CI 50325), Acid Blue 1 (Patent Blue, CI 42045), Acid Blue 3 (Patent Blue V, CI 42051), Acid Blue 7 (CI 42080), Acid Blue 104 (CI 42735), Acid Blue 9 (E 133, Patentblau AE, Amidoblau AE, Erioglaucin A, CI 42090, CI Food Blue 2), Acid Blue 62 (CI 62045), Acid Blue 74 (E 132, CI 73015), Acid Blue 80 (CI 61585), Acid Green 3 (CI 42085, Foodgreen1), Acid Green 5 (CI 42095), Acid Green 9 (CI42100), Acid Green 22 (CI42170), Acid Green 25 (CI 61570, Japan Green 201, D&C Green no. 5), Acid Green 50 (Brilliant Acid Green BS, CI 44090, Acid Brilliant Green BS, E 142), Acid Black 1 (Black no 401, Naphthalene Black 10B, Amido Black 10B, CI 20 470, COLIPA no B15), Acid Black 52 (CI 15711), Food Yellow 8 (CI 14270), Food Blue 5, D&C Yellow 8, D&C Green 5, D&C Orange 10, D&C Orange 11, D&C Red 21, D&C Red 27, D&C Red 33, D&C Violet 2 and/or D&C Brown 1.

The water solubility of the anionic direct dyes can be determined, for example, in the following manner. 0.1 g of the anionic direct dye is placed in a beaker. A stirring bar is added. Then 100 ml of water are added. This mixture is heated to 25° C. on a magnetic stirrer, while stirring. The mixture is stirred for 60 minutes. Thereafter, the aqueous mixture is visually assessed. If there are still undissolved residues, the amount of water is increased, for example in steps of 10 ml. Water is added until the amount of dye used has dissolved completely. If the dye-water mixture cannot be visually assessed due to the high intensity of the dye, the mixture is filtered. If a proportion of undissolved dyes remains on the filter paper, the solubility test is repeated with a larger amount of water. If 0.1 g of the anionic direct dye dissolves in 100 ml of water at 25° C., then the solubility of the dye is 1.0 g/L.

Acid Yellow 1 carries the name 8-hydroxy-5,7-dinitro-2-naphthalenesulfonic acid disodium salt and has a solubility in water of at least 40 g/L (25° C.).

Acid Yellow 3 is a mixture of the sodium salts of mono- and sis-sulfonic acids of 2-(2-quinolyl)-1H-indene-1,3(2H)-dione and has a water solubility of 20 g/L (25° C.).

Acid Yellow 9 is the disodium salt of 8-hydroxy-5,7-dinitro-2-naphthalenesulfonic acid, its water solubility is above 40 g/L (25° C.).

Acid Yellow 23 is the trisodium salt of 4,5-dihydro-5-oxo-1-(4-sulfophenyl)-4-((4-sulfophenyl)azo)-1H-pyrazole-3-carboxylic acid, and is readily soluble in water at 25° C.

Acid Orange 7 is the sodium salt of 4-[(2-hydroxy-1-naphthyl)azo]benzene sulphonate. Its water solubility is more than 7 g/L (25° C.).

Acid Red 18 is the trisodium salt of 7-hydroxy-8-[(E)-(4-sulfonato-1-naphthyl)diazenyl)]-1,3-naphthalenedisulfonate and has a very high water solubility of more than 20 wt. %.

Acid Red 33 is the disodium salt of 5-amino-4-hydroxy-3-(phenylazo)-naphthalene-2,7-disulfonate, its water solubility is 2.5 g/L (25° C.).

Acid Red 92 is the disodium salt of 3,4,5,6-tetrachloro-2-(1,4,5,8-tetrabromo-6-hydroxy-3-oxanthene-9-yl) benzoic acid, the water solubility of which is specified as greater than 10 g/L (25° C.).

Acid Blue 9 is the disodium salt of 2-({4-[N-ethyl(3-sulfonatobenzyl]amino] phenyl} {4-[(N-ethyl(3-sulfonatobenzyl)imino]-2,5-cyclohexadien-1-ylidene}methyl)-benzenesulfonate and has a water solubility of more than 20 wt. % (25° C.).

Furthermore, thermochromic dyes can also be used. Thermochromism involves the property of reversibly or irreversibly changing the color of a material depending on the temperature. This can take place both by changing the intensity and/or by changing the wavelength maximum.

Finally, it is also possible to use photochromic dyes. Photochromism involves the property of reversibly or irreversibly changing the color of a material, depending on the irradiation with light, in particular UV light. This can take place both by changing the intensity and/or by changing the wavelength maximum.

### Water Content of Composition (B)

The composition (B) preferably contains the dyeing compound(s) (B1) in a cosmetic carrier, particularly preferably in an aqueous cosmetic carrier.

In this context, it has been found to be preferred if the composition (B) contains, based on the total weight of the composition (B), 5.0 to 90.0 wt. %, preferably 30.0 to 98.0 wt. %, preferably 40.0 to 95.0 wt. %, more preferably 45.0 to 90.0 wt. %, even more preferably 50.0 to 90.0 wt. %, and very particularly preferably 55.0 to 90.0 wt. % water.

Within the context of a further embodiment, a method according to the invention is characterized in that the composition (B) contains, based on the total weight of the composition (B), 30.0 to 98.0 wt. %, preferably 40.0 to 95.0 wt. %, more preferably 45.0 to 90.0 wt. %, even more preferably 50.0 to 90.0 wt. %, and very particularly preferably 55.0 to 90.0 wt. % water.

### Further Cosmetic Ingredients in the Composition (B)

In addition, the composition (B) can furthermore also contain one or more additional cosmetic ingredients.

The cosmetic ingredients which can optionally be used in the composition (B) can be all suitable components for giving the agent further positive properties. For example, the composition (B) can contain a solvent, a surface-active compound from the group of the non-ionic, cationic, anionic or zwitterionic/amphoteric surfactants, the dyeing compounds from the group of the pigments, the direct dyes, the film-forming polymers, the fatty components from the group of the C₈-C₃₀ fatty alcohols, the hydrocarbon compounds, fatty acid esters, the acids and bases belonging to the group of the pH regulators, the perfumes, the preservatives and the plant extracts.

The selection of these additional substances is made by a person skilled in the art according to the desired properties of the agents. With regard to other optional components and the amounts of said components used, reference is explicitly made to relevant reference books known to a person skilled in the art.

### Film-Forming Polymers

In order to increase the fastness to washing, the composition (A) and/or the composition (B) can additionally also contain at least one film-forming polymer as an optional component.

Within the context of a further embodiment, a method according to the invention is characterized in that the composition (A) and/or the composition (B) contains at least one film-forming polymer.

Polymers are to be understood as macromolecules having a molecular weight of at least 1,000 g/mol, preferably at least 2,500 g/mol, particularly preferably at least 5,000 g/mol, which consist of like, repeating organic units. The polymers of the present invention may be synthetically prepared polymers prepared by polymerization of a monomer type or by polymerization of various structurally different monomer types. If the polymer is prepared by polymerizing one type of monomer, it is a homopolymer. If structurally different monomer types are used in the polymerization, the resultant polymer is referred to as a copolymer.

The maximum molecular weight of the polymer depends on the degree of polymerization (number of polymerized monomers) and the batch size, and is also determined by the polymerization method. Within the meaning of the present invention, it is preferable if the maximum molecular weight of the film-forming hydrophobic polymer (c) is not more than 107 g/mol, preferably not more than 106 g/mol, and particularly preferably not more than 105 g/mol.

Within the meaning of the invention, a film-forming polymer is understood to mean a polymer which is capable of forming a film on a substrate, for example on a keratin material or a keratin fiber. The formation of a film can be determined, for example, by observing the keratin material treated with the polymer under a microscope.

The film-forming polymers may be hydrophilic or hydrophobic.

Within the context of a further embodiment, it may be preferred to use at least one hydrophobic film-forming polymer in the composition (B).

A hydrophobic polymer is understood to mean a polymer that has a solubility in water at 25° C.(760 mmHg) of less than 1 wt. %.

The water solubility of the film-forming hydrophobic polymer can be determined, for example, in the following manner. 1.0 g of the polymer is placed in a beaker. Water is added up to 100 g. A stirring bar is added, and the mixture is heated to 25° C. on a magnetic stirrer, while stirring. The mixture is stirred for 60 minutes. Thereafter, the aqueous mixture is visually assessed. If the polymer-water mixture cannot be visually assessed due to a high turbidity of the mixture, the mixture is filtered. If a proportion of undissolved polymer remains on the filter paper, the solubility of the polymer is less than 1 wt. %.

Here, the polymers of the acrylic acid type, the polyurethanes, the polyesters, the polyamides, the polyureas, the nitro-cellulose polymers, the silicone polymers, the polymers of the acrylamide type and the polyisoprenes can be mentioned in particular.

Particularly well-suited film-forming, hydrophobic polymers are, for example, polymers from the group of the copolymers of acrylic acid, the copolymers of methacrylic acid, the homopolymers or copolymers of acrylic acid esters, the homopolymers or copolymers of methacrylic acid esters, the homopolymers or copolymers of acrylic acid amides, the homopolymers or copolymers of methacrylic acid amides, the copolymers of vinylpyrrolidone, the copolymers of vinyl alcohol, the copolymers of vinyl acetate, the homopolymers or copolymers of ethylene, the homopolymers or copolymers of propylene, the homopolymers or copolymers of styrene, the polyurethanes, the polyesters and/or the polyamides.

In particular the film-forming hydrophobic polymers which are selected from the group of the synthetic polymers, the polymers obtainable by free-radical polymerization, or the natural polymers, have proven particularly well-suited for achieving the object according to the invention.

Further particularly well-suited film-forming hydrophobic polymers can be selected from the homopolymers or copolymers of olefins, for example cycloolefins, butadiene, isoprene or styrene, vinyl ethers, vinylamides, the esters or amides of (meth)acrylic acid having at least one C₁-C₂₀ alkyl group, an aryl group, or a C₂-C₁₀ hydroxyalkyl group.

Further film-forming hydrophobic polymers may be selected from the homo- or copolymers of isooctyl(meth)acrylate; isononyl(meth)acrylate; 2-ethylhexyl(meth)acrylate; lauryl(meth)acrylate); isopentyl(meth)acrylate; n-butyl(meth)acrylate); isobutyl(meth)acrylate; ethyl(meth)acrylate; tert-butyl(meth)acrylate; stearyl(meth)acrylate; methyl(meth)acrylate; hydroxyethyl(meth)acrylate; 2-hydroxypropyl(meth)acrylate; 3-hydroxypropyl(meth)acrylate, and/or mixtures thereof.

Further film-forming hydrophobic polymers can be selected from the homo- or copolymers of (meth)acrylamide; N-alkyl(meth)acrylamides, in particular those having C2-C18 alkyl groups, for example N-ethyl acrylamide, N-tert-butyl acrylamide, N-octyl crylamide; N-Di(C1-C4)alkyl(meth)acrylamide.

Further preferred anionic copolymers are, for example, copolymers of acrylic acid, methacrylic acid or the C₁-C₆ alkyl esters thereof, such as are marketed under the INCI declaration acrylates copolymer. A suitable commercial product is for example Aculyn® 33 by the company Rohm & Haas. However, copolymers of acrylic acid, methacrylic acid or their C₁-C₆ alkyl esters and the esters of an ethylenically unsaturated acid and an alkoxylated fatty alcohol are further preferred. Suitable ethylenically unsaturated acids are in particular acrylic acid, methacrylic acid and itaconic acid; suitable alkoxylated fatty alcohols are in particular Steareth-20 or Ceteth-20.

Very particularly preferred polymers on the market are, for example, Aculyn® 22 (Acrylates/Steareth-20 Methacrylate Copolymer), Aculyn® 28 (Acrylates/Beheneth-25 Methacrylate Copolymer), Structure 2001® (Acrylates/Steareth-20 Itaconate Copolymer), Structure 3001® (Acrylates/Ceteth-20 Itaconate Copolymer), Structure Plus® (Acrylates/Aminoacrylates C10-30 Alkyl PEG-20 Itaconate Copolymer), Carbopol® 1342, 1382, Ultrez 20, Ultrez 21 (Acrylates/C10-30 Alkyl Acrylate Crosspolymer), Synthalen W 2000® (Acrylates/Palmeth-25 Acrylate Copolymer) or Soltex OPT (Acrylates/C12-22 Alkyl methacrylate Copolymer) distributed by Rohme & Haas.

Examples of suitable polymers based on vinyl monomers are the homopolymers and copolymers of N-vinylpyrrolidone, vinylcaprolactam, the Vinyl (C1-C6) alkyl pyrrole, vinyl oxazoline, vinyl thiazole, vinylpyrimidine, vinylimidazole.

Also very particularly suitable are the copolymers octylacrylamide/acrylates/butylamino-ethyl-methacrylate copolymer, as is commercially marketed, for example, under the trade names AMPHOMER® or LOVOCRYL® 47 by NATIONAL STARCH, or the copolymers of acrylates/octylacrylamides which are marketed by NATIONAL STARCH under the trade names DERMACRYL® LT and DERMACRYL® 79.

Examples of suitable polymers based on olefins are the homo- and copolymers of ethylene, propylene, butene, isoprene and butadiene.

Within the context of a further embodiment, the block copolymers which comprise at least one block of styrene or the derivatives of styrene can be used as film-forming hydrophobic polymers. These block copolymers can be copolymers which, in addition to a styrene block, contain one or more further blocks, such as styrene/ethylene, styrene/ethylene/butylene, styrene/butylene, styrene/isoprene, styrene/butadiene. Corresponding polymers are commercially available from BASF under the trade name “Luvitol HSB”.

Within the context of a further embodiment, it may be preferred to use at least one hydrophilic film-forming polymer in the composition (A) and/or in the composition (B).

A hydrophilic polymer is understood to mean a polymer that has a solubility in water at 25° C. (760 mmHg) of more than 1 wt. %, preferably of more than 2 wt. %.

The water solubility of the film-forming hydrophilic polymer can be determined, for example, in the following manner. 1.0 g of the polymer is placed in a beaker. Water is added up to 100 g. A stirring bar is added, and the mixture is heated to 25° C. on a magnetic stirrer, while stirring. The mixture is stirred for 60 minutes. Thereafter, the aqueous mixture is visually assessed. A completely dissolved polymer appears macroscopically homogeneous. If the polymer-water mixture cannot be visually assessed due to a high turbidity of the mixture, the mixture is filtered. If no undissolved polymer remains on the filter paper, the solubility of the polymer is more than 1 wt. %.

Non-ionic, anionic and cationic polymers can be used as film-forming hydrophilic polymers.

Suitable film-forming hydrophilic polymers can be selected, for example, from the group of the polyvinylpyrrolidone (co)polymers, the polyvinyl alcohol (co)polymers, the vinyl acetate (co)polymers, the carboxyvinyl (co)polymers, the acrylic acid (co)polymers, the methacrylic acid (co)polymers, the natural gums, the polysaccharides and/or the acrylamide (co)polymers.

Furthermore, it is very particularly preferred to use polyvinylpyrrolidone (PVP) and/or a copolymer containing vinylpyrrolidone as the film-forming hydrophilic polymer.

It is further preferred if the composition (A) and/or (B) according to the invention contains polyvinylpyrrolidone (PVP) as the film-forming hydrophilic polymer. Surprisingly, the fastness to washing, of the dyes which could be obtained with PVP-containing agents, was also very good.

Particularly well-suited polyvinylpyrrolidones are available, for example, under the name Luviskol® K from BASF SE, in particular Luviskol® K 90 or Luviskol® K 85 from BASF SE.

The polymer PVP K30, which is sold by Ashland (ISP, POI Chemical) can also be used as further explicitly very particularly suitable polyvinylpyrrolidone (PVP). PVP K 30 is a polyvinylpyrrolidone which is very soluble in cold water and which has the CAS number 9003-39-8. The molar weight of PVP K 30 is about 40,000 g/mol.

Further very particularly suitable polyvinylpyrrolidones are the substances known under the trade names LUVITEC K 17, LUVITEC K 30, LUVITEC K 60, LUVITEC K 80, LUVITEC K 85, LUVITEC K 90 and LUVITEC K 115 and available from BASF.

The use of film-forming hydrophilic polymers from the group of copolymers of the polyvinylpyrrolidone likewise has led to particularly good and wash-resistant dyeing results.

In this context, vinylpyrrolidone-vinyl ester copolymers, as are marketed, for example, under the trademark Luviskol® (BASF), can be cited as particularly well-suited film-forming hydrophilic polymers. Luviskol® VA 64 and Luviskol® VA 73, each being vinylpyrrolidone/vinyl acetate copolymers, are particularly preferred non-ionic polymers.

Of the vinylpyrrolidone-containing copolymers, a styrene/VP copolymer and/or a vinylpyrrolidone-vinyl acetate copolymer and/or a VP/DMAPA acrylates copolymer and/or a VP/vinyl caprolactam/DMAPA acrylates copolymer are very particularly preferably used in the cosmetic compositions.

Vinylpyrrolidone-vinyl acetate copolymers are marketed under the name Luviskol® VA by BASF SE. A VP/vinyl caprolactam/DMAPA acrylates copolymer is marketed, for example, under the trade name Aquaflex® SF-40 by Ashland Inc. A VP/DMAPA acrylates copolymer is marketed, for example, under the name Styleze CC-10 by Ashland, and is a highly preferred vinylpyrrolidone-containing copolymer.

As further suitable copolymers of polyvinylpyrrolidone, the copolymers which are obtained by reacting N-vinylpyrrolidone with at least one further monomer from the group consisting of V-vinylformamide, vinyl acetate, ethylene, propylene, acrylamide, vinylcaprolactam, vinyl caprolactone and/or vinyl alcohol can also be mentioned.

A further suitable copolymer of vinylpyrrolidone is the polymer known under the INCI name Maltodextrin/VP Copolymer.

Furthermore, intensively dyed keratin material, in particular hair, was obtained with very good fastness to washing when a non-ionic, film-forming, hydrophilic polymer was used as the film-forming hydrophilic polymer.

According to a further embodiment, it may be preferred if the composition (B) contains at least one non-ionic, film-forming, hydrophilic polymer.

According to the invention, a non-ionic polymer is understood to be a polymer that bears, in a protic solvent, such as water, under standard conditions, substantially no structural units having permanently cationic or anionic groups, which must be compensated by counterions so as to maintain the electroneutrality. Quaternized ammonium groups, for example, fall under cationic groups, but protonated amines do not. Carboxyl groups and sulfonic acid groups, for example, fall under anionic groups.

The agents are very particularly preferred which contain, as the non-ionic, film-forming hydrophilic polymer, at least one polymer selected from the group consisting of
polyvinylpyrrolidone,
copolymers of N-vinylpyrrolidone and vinyl esters of carboxylic acids having from 2 to 18 carbon atoms, in particular of N-vinylpyrrolidone and vinyl acetate,
copolymers of N-vinylpyrrolidone and N-vinylimidazole and methacrylamide,
copolymers of N-vinylpyrrolidone and N-vinylimidazole and acrylamide,
copolymers of N-vinylpyrrolidone with N,N-di(C1 to C4)-alkylamino-(C2 to C4)-alkyl acrylamide.

If copolymers of N-vinylpyrrolidone and vinyl acetate are used, it is again preferred if the molar ratio of the structural units comprised of the monomer N-vinylpyrrolidone to the structural units comprised of the monomer vinyl acetate is in the range from 20 to 80 to 80 to 20, in particular from 30 to 70 to 60 to 40. Suitable copolymerisates of vinylpyrrolidone and vinyl acetate are available, for example, under the trademark Luviskol® VA 37, Luviskol® VA 55, Luviskol® VA 64 and Luviskol® VA 73, from the company BASF SE.

In this case, a further particularly preferred polymer is selected from the polymers having the INCI name VP/methacrylamide/vinyl imidazole copolymer, which are available, for example, from BASF SE under the trade name Luviset Clear.

A further very particularly preferred non-ionic film-forming hydrophilic polymer is a copolymer of N-vinylpyrrolidone and N,N-dimethylaminopropylmethacrylamide, which, for example, is sold by the company ISP under the INCI name VP/DMAPA Acrylates Copolymer, for example under the trade name Styleze® CC 10.

A cationic polymer according to the invention is the copolymer of N-vinylpyrrolidone, N-vinylcaprolactam, N-(3-dimethylaminopropyl)methacrylamide and 3-(methacryloylamino)propyl-lauryl-dimethylammonium chloride (INCI name: Polyquaternium-69), which is marketed, for example, under the trade name AquaStyle® 300 (28-32 wt. % active substance in an ethanol-water mixture, molecular weight 350000) by the company ISP.

Further suitable film-forming hydrophilic polymers are, for example,
vinylpyrrolidone-vinylimidazolium methochloride copolymers, as are offered under the designations Luviquat® FC 370, FC 550 and the INCI designation Polyquaternium-16, as well as FC 905 and HM 552,
vinylpyrrolidone-vinylcaprolactam acrylate terpolymers, as are offered for example under the name Aquaflex® SF 40, having acrylic acid esters and acrylic acid amides as a third monomer unit.

Polyquaternium-11 is the reaction product of diethyl sulfate having a copolymer of vinylpyrrolidone and dimethylaminoethyl methacrylate. Suitable commercial products are available, for example, under the names Dehyquart® CC 11 and Luviquat® PQ 11 PN from the company BASF SE or Gafquat 440, Gafquat 734, Gafquat 755 or Gafquat 755N by Ashland Inc.

Polyquaternium-46 is the reaction product of vinylcaprolactam and vinylpyrrolidone with methylvinylimidazolium methosulfate and is available, for example, under the name Luviquat® Hold from BASF SE. Polyquaternium-46 is preferably used in an amount of 1 to 5 wt. %, based on the total weight of the cosmetic composition. It is very particularly preferred that Polyquaternium-46 is used in combination with a cationic guar compound. It is even most preferred that Polyquaternium-46 is used in combination with a cationic guar compound and Polyquaternium-11.

For example, acrylic acid polymers which may be present in uncrosslinked or crosslinked form can be used as suitable anionic film-forming hydrophilic polymers. Corresponding products are marketed commercially for example under the trade names Carbopol 980, 981, 954, 2984 and 5984 by the company Lubrizol or also under the names Synthalen M and Synthalen K by the company 3V Sigma (The Sun Chemicals, Inter Harz).

Examples of suitable film-forming, hydrophilic polymers from the group of the natural gums are xanthan gum, gellan gum, carob gum.

Suitable film-forming hydrophilic polymers from the group of the acrylamides are, for example, polymers which are prepared proceeding from monomers of the (meth)acrylamido-C₁-C₄-alkylsulfonic acids or the salts thereof. Corresponding polymers can be selected from the polymers of polyacrylamidomethanesulfonic acid, polyacrylamidoethanesulfonic acid, polyacrylamidopropanesulfonic acid, poly-2-acrylamido-2-methylpropanesulfonic acid, poly-2-methylacrylamido-2-methylpropanesulfonic acid and/or poly-2-methylacrylamido-n-butanesulfonic acid.

Preferred polymers of the poly(meth)arylamido-C₁-C₄-alkylsulfonic acids are crosslinked and at least 90% neutralized. These polymers can be crosslinked or also uncrosslinked.

Crosslinked and completely or partially neutralized polymers of the type of the poly-2-acrylamido-2-methylpropanesulfonic acids are known under the INCI names “Ammonium Polyacrylamido-2-methyl-propanesulphonate” or “Ammonium Polyacryldimethyltauramide”.

Another preferred polymer of this type is the cross-linked poly-2-acrylamido-2-methyl-propaneulphonic acid polymer marketed by the company Clamant under the trade name Hostacerin AMPS, which is partially neutralized with ammonia.

In a further explicitly very particularly preferred embodiment, a method according to the invention is characterized in that the composition (A) and/or the composition (B) contains at least one anionic, film-forming polymer.

In this context, the best results could be obtained when the composition (A) and/or the composition (B) contains at least one film-forming polymer which comprises at least one structural unit of formula (P-I) and at least one structural unit of formula (P-II)

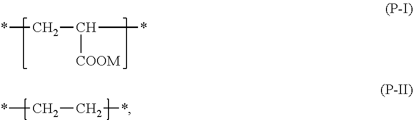

in which
M represents a hydrogen atom or ammonium (NH₄), sodium, potassium, ½ magnesium or ½ calcium.
If M represents a hydrogen atom, the structural unit of formula (P-I) is based on an acrylic acid unit.
If M represents an ammonium counterion, the structural unit of formula (P-I) is based on the ammonium salt of acrylic acid.
If M represents a sodium counterion, the structural unit of formula (P-I) is based on the sodium salt of acrylic acid.
If M represents a potassium counterion, the structural unit of formula (P-I) is based on the potassium salt of acrylic acid.
If M represents a half equivalent of a magnesium counterion, the structural unit of formula (P-I) is based on the magnesium salt of acrylic acid.
If M represents a half equivalent of a calcium counterion, the structural unit of formula (P-I) is based on the calcium salt of acrylic acid.

The film-forming polymer(s) according to the invention are preferably used in specific quantity ranges in the respective composition. In this context, it has proven to be particularly preferred, in order to achieve the object according to the invention if the composition contains, based in each case on the total weight thereof, one or more film-forming polymers in a total amount of 0.1 to 18.0 wt. %, preferably 1.0 to 16.0 wt. %, more preferably 5.0 to 14.5 wt. %, and very particularly preferably 8.0 to 12.0 wt. %.

### Application of Compositions (A) and (B) in the Keratin Treatment Method

The method according to the invention comprises the application of the two compositions (A) and (B) to the keratin material. The two compositions (A) and (B) are in the two different compositions.

Within the context of one embodiment, it may be preferred to mix the compositions (A) and (B) with one another before application to the keratin material, such that the mixture of (A) and (B) is applied to the keratin material.

Within the context of a further particularly preferred embodiment, a method according to the invention is characterized in that the compositions (A) and (B) are mixed with one another and their blend is applied to the keratin material.

For example, the compositions (A) and (B) can be stirred or shaken together shortly before application, or mixed with one another in some other way. The mixing can take place, for example, by transferring the composition (A) from the container (A), in which it has been made available to the user, into a container (B), the composition (B) being located in the container (B). Alternatively, the composition (B) is transferred from the container (B), in which it has been made available to the user, into a container (A), the composition (A) being located in the container (A). The application mixture of (A) and (B), prepared in this way, can then be applied to the keratin material for example within 1 to 240 minutes, preferably within 1 to 180 minutes, more preferably within 1 to 120 minutes, after preparation thereof.

Within the context of a further embodiment, a method according to the invention is particularly preferred which comprises the following steps:
(1) preparing an application mixture by mixing the compositions (A) and (B)
(2) applying the mixture of (A) and (B) to the keratin material,
(3) allowing the mixture of (A) and (B) to act on the keratin material for a period of 1 to 10 minutes, preferably 1 to 5 minutes,
(4) rinsing out the mixture from the keratin material.

Furthermore, the successive application of the compositions (A) and (B), i.e. in this case the composition (A) can preferably first be applied to the keratin material, allowed to act, and optionally rinsed out again, is also possible and also in accordance with the invention. Thereafter, the composition (B) is then applied to the keratin material, allowed to act and optionally rinsed out again.

Within the context of a further particularly preferred embodiment, a method according to the invention is characterized in that the compositions (A) and (B) are applied one after the other to the keratin material.

Within the context of this further embodiment, a method according to the invention is characterized by the following steps:
(1) applying the first composition (A) to the keratin material,
(2) allowing the composition (A) to act on the keratin material for a period of 1 to 10 minutes, preferably 1 to 5 minutes,
(3) rinsing out the composition (A) from the keratin material,
(4) applying the composition (B) to the keratin material,
(5) allowing the composition (B) to act on the keratin material for a period of 1 to 10 minutes, preferably 1 to 5 minutes,
(6) rinsing out the composition (B) from the keratin material.

The rinsing out of the keratin material with water in steps (3) and (6) of the method is understood, according to the invention, to mean that only water is used for the rinsing process, without any further composition, different from the compositions (A) and (B), being used.

In step (1), the composition (A) is first applied to the keratin materials, in particular human hair.

After application, the composition (A) is allowed to act on the keratin materials. In this context, application times of 10 seconds to 10 minutes, preferably of 20 seconds to 5 minutes and very particularly preferably of 30 seconds to 2 minutes on the hair have proven to be particularly advantageous.

Within the context of a preferred embodiment of the method according to the invention, the composition (A) can now be rinsed out of the keratin materials before the composition (B) is applied to the hair in the subsequent step.

In step (4), the composition (B) is now applied to the keratin materials. After application, the composition (B) is now allowed to act on the hair.

The method according to the invention allows the creation of dyes having particularly good intensity and fastness to washing, even in the case of a short application time of the compositions (A) and (B). Application times of 10 seconds to 10 minutes, preferably of 20 seconds to 5 minutes, and very particularly preferably of 30 seconds to 3 minutes on the hair have proven to be particularly advantageous.

In step (6), the composition (B) is now rinsed out of the keratin material with water.

Within the context of a further embodiment, a method according to the invention comprises the following steps in the stated sequence:
(1) applying the first composition (A) to the keratin material,
(2) allowing the composition (A) to act on the keratin material for a period of 1 to 10 minutes, preferably 1 to 5 minutes,
(3) no rinsing out of the composition (A) from the keratin material,
(4) applying the composition (B) to the keratin material which is still exposed to the composition (A),
(5) allowing the composition (B) to act on the keratin material for a period of 1 to 10 minutes, preferably 1 to 5 minutes,
(6) rinsing out compositions (A) and (B) from the keratin material.

### Heat Treatment of the Keratin Material

In addition to the application of compositions (A) and (B), the method according to the invention is characterized in that the keratin material, in particular human hair, undergoes heat treatment.

Heat treatment is understood to mean that the keratin material is brought into contact with a heated appliance, or that this heated device is applied on or to the keratin material. Furthermore, the keratin material can also be brought into contact with warm/hot air for the heat treatment. For example, a hair dryer, a blow-dryer, a thermal hood, a straightening iron, a curling iron or an infrared lamp may be used as the appliance.

Within the context of a particularly preferred embodiment, a method according to the invention is characterized in that the heat treatment is carried out by applying a hair dryer, a blow-dryer, a thermal hood, a straightening iron, a curling iron or an infrared lamp.

It has also been found that it is preferred for the treatment temperature during the heat treatment to be between 40° C. to 210° C., preferably from 50° C. to 190° C., more preferably from 50° C. to 170° C., even more preferably from 50° C. to 150° C. and very particularly preferably from 50° C. to 100° C. In other words, it has been found to be particularly preferred if the heat treatment is carried out using an appliance which is heated to a temperature of 40° C. to 210° C., preferably 50° C. to 190° C., more preferably 50° C. to 170° C., even more preferably 50° C. to 150° C., and very particularly preferably 50° C. to 100° C.

Within the context of a particularly preferred embodiment, a method according to the invention is characterized in that the heat treatment is carried out using an appliance which is heated to a temperature of 40° C. to 210° C., preferably 50° C. to 190° C., more preferably 50° C. to 170° C., even more preferably 50° C. to 150° C., and very particularly preferably 50° C. to 100° C.

For example, the keratin material or the hair can thus be treated with a blow-dryer that blows warm or hot air onto the keratin material. This air is very particularly preferably 50 to 100° C. Alternatively, the keratin material or the hair is held under an infrared lamp, which is particularly preferably set to a temperature of 50 to 100° C. For the purpose of heat treatment, hair can also be pressed between two correspondingly temperature-controlled plates of a straightening iron, it being possible for the plates to be moved simultaneously along the fiber. The plates of the straightening iron can be set, for example, to a temperature of up to 210° C.

The duration of the heat treatment can be adjusted to the selected temperature range. For example, a heat treatment can be carried out over a period of 5 seconds to 60 minutes, preferably 15 seconds to 45 minutes, more preferably 15 seconds to 30 minutes, and very particularly preferably 15 seconds to 15 minutes.

The work leading to this invention has shown that it is particularly preferred if the heat treatment takes place after application of the composition (A) and/or after application of the composition (B).

Within the context of a particularly preferred embodiment, a method according to the invention is characterized in that the keratin material, which is exposed to the composition (A) and/or to the composition (B), undergoes a heat treatment.

Within the context of a preferred embodiment, keratin material is treated which is still exposed to the composition (A).

Within the context of a further preferred embodiment, the heat treatment takes place after the application and rinsing out of the composition (A), but before the application of the composition (B).

Within the context of a further preferred embodiment, keratin material is treated which is still exposed to the composition (B).

Within the context of a further preferred embodiment, the heat treatment takes place after both compositions (A) and (B)—either simultaneously or successively—have been applied to the keratin material and rinsed out again.

A method according to the invention is very particularly preferred which comprises the following steps in the stated sequence:
(1) providing compositions (A) and (B),
(2) preparing a mixture of the compositions (A) and (B),
(3) applying the blend of (A) and (B) to the keratin material,
(4) the blend of (A) and (B) acting on the keratin material,
(5) optionally rinsing out both compositions (A) and (B), and
(6) heat treatment of the keratin material, preferably of the still damp keratin material.

A method according to the invention is very particularly preferred which comprises the following steps in the stated sequence:
(1) applying the composition (A) to the keratin material,
(2) the composition (A) acting on the keratin material,
(3) optionally rinsing out the composition (A),
(4) heat treatment of the keratin material, preferably of the still damp keratin material,
(5) applying the composition (B) to the keratin material,
(6) the composition (B) acting on the keratin material, and
(7) optionally rinsing out the composition (B).

A method according to the invention is also very particularly preferred which comprises the following steps in the stated sequence:
(1) applying the composition (A) to the keratin material,
(2) the composition (A) acting on the keratin material,
(3) optionally rinsing out the composition (A),
(4) applying the composition (B) to the keratin material,
(5) the composition (B) acting on the keratin material,
(6) optionally rinsing out the composition (B), and
(7) heat treatment of the keratin material, preferably of the still damp keratin material.

The composition (A), the composition (B) and the heat treatment have already been disclosed in detail above.

Since steps (1) to (6) or (1) to (7) are carried out within a keratin treatment method, a period of at most 24 hours, preferably at most 12 hours, more preferably at most 6 hours, and most preferably at most 3 hours exists between the performance of step (1) and the performance of step (6) (or (7)).

In the course of the method according to the invention, the keratin material can both be subjected completely to heat treatment, but also the treatment of partial regions of the keratin material can be included. The complete heat treatment of the keratin material is preferred, i.e. preferably all the keratin material to which the compositions (A) and (B) were also applied, is treated with heat.

1. A method for dyeing keratin material, wherein the method comprises:
applying a first composition to the keratin material, the first composition comprising one or more organic C₁-C₆ alkoxy silanes, the condensation products thereof, or mixtures thereof;
applying a second composition to the keratin material, the second composition comprising one or more dyeing compounds comprising pigments, direct dyes, or mixtures thereof; and
applying a heat treatment to the keratin material.

2. The method according to claim 1, wherein the one or more organic C₁-C₆ alkoxy silanes are represented by formula (S-I) and/or (S-II),
R₁R₂N-L-Si(OR₃)ₐ(R₄)_{b} (S-I),
wherein:
R₁, R₂ represent, independently of one another, a hydrogen atom or a C₁-C₆ alkyl group,
L represents a linear or branched, divalent C₁-C₂₀ alkylene group,
R₃, R₄ represent, independently of one another, a C₁-C₆ alkyl group,
a represents an integer from 1 to 3, and
b represents the integer 3-a, and
(R₅O)_{c}(R₆)_{d}Si-(A)ₑ-[NR₇-(A′)]_{f}-[O-(A″)]_{g}-[NR₈-(A′″)]ₕ-Si(R₆′)_{d′}(OR₅′)_{c′} (S-II),
wherein:
R₅, R₅′, R₅″, R₆, R₆′ and R₆″ represent, independently of one another, a C₁-C₆ alkyl group,
A, A′, A″, A′″ and A″″ represent, independently of one another, a linear or branched, divalent C₁-C₂₀ alkylene group,
R₇ and R₈ represent, independently of one another, a hydrogen atom, a C₁-C₆ alkyl group, a hydroxy C₁-C₆ alkyl group, a C₂-C₆ alkenyl group, an amino C₁-C₆ alkyl group, or a group of formula (S-III),
(A″″)-Si(R₆″)_{d}″(OR₅″)_{c}″ (S-III),
c represents an integer from 1 to 3,
d represents the integer 3-c,
c′ represents an integer from 1 to 3,
d′ represents the integer 3-c′,
c″ represents an integer from 1 to 3,
d″ represents the integer 3-c″,
e represents 0 or 1,
f represents 0 or 1,
g represents 0 or 1,
h represents 0 or 1,
wherein at least one of the groups e, f, g and h is different from 0,
and/or the condensation products thereof.

3. The method according to claim 1, wherein the first composition comprises at least one organic C₁-C₆ alkoxy silane selected from the group consisting of:
(3-aminopropyl)triethoxysilane,
(3-aminopropyl)trimethoxysilane,
(2-aminoethyl)triethoxysilane,
(2-aminoethyl)trimethoxysilane,
(3-dimethylaminopropyl)triethoxysilane,
(3-dimethylaminopropyl)trimethoxysilane,
(2-dimethylaminoethyl)triethoxysilane,
(2-dimethylaminopropyl)trimethoxysilane,
condensation products thereof; and
mixtures thereof.

4. The method according to claim 1, wherein the first composition comprises one or more organic C₁-C₆ alkoxy silanes represented by formula (S-IV),
R₉Si(OR₁₀)ₖ(R₁₁)ₘ (S-IV),
wherein:
R₉ represents a C₁-C₁₂ alkyl group,
R₁₀ represents a C₁-C₆ alkyl group,
R₁₁ represents a C₁-C₆ alkyl group,
k represents an integer from 1 to 3, and
m represents the integer 3-k,
and/or the condensation products thereof.

5. The method according to claim 1, wherein the first composition comprises at least one organic C₁-C₆ alkoxy silane selected from the group consisting of:
methyltrimethoxysilane,
methyltriethoxysilane,
ethyltrimethoxysilane,
ethyltriethoxysilane,
hexyltrimethoxysilane,
hexyltriethoxysilane,
octyltrimethoxysilane,
octyltriethoxysilane,
dodecyltrimethoxysilane,
dodecyltriethoxysilane,
and/or the condensation products thereof; and
mixtures thereof.

6. The method according to claim 2, wherein the first composition comprises at least one organic C₁-C₆ alkoxy silane represented by formula (S-I) and at least one organic C₁-C₆ alkoxy silane (A1) of formula (S-IV),
R₉Si(OR₁₀)ₖ(R₁₁)ₘ (S-IV),
wherein:
R₉ represents a C₁-C₁₂ alkyl group,
R₁₀ represents a C₁-C₆ alkyl group,
R₁₁ represents a C₁-C₆ alkyl group,
k represents an integer from 1 to 3, and
m represents the integer 3-k.

7. The method according to claim 1, wherein the first composition comprises, based on the total weight of the first composition, one or more organic C₁-C₆ alkoxy silanes, the condensation products thereof, or mixtures thereof in a total amount ranging from 1.0 to 99.0 wt. %.

8. The method according to claim 1, wherein the second composition comprises at least one inorganic pigment selected from the group consisting of colored metal oxides, metal hydroxides, metal oxide hydrates, silicates, metal sulfides, complex metal cyanides, metal sulphates, bronze pigments and/or from mica-based colored pigments which are coated with at least one metal oxide and/or metal oxychloride, and combinations thereof.

9. The method according to claim 1, wherein the second composition comprises at least one organic pigment selected from the group consisting of:
carmine;
quinacridone;
phthalocyanine;
sorghum;
blue pigments having the Color Index Numbers C1 42090, CI 69800, CI 69825, CI 73000, CI 74100, CI 74160, or combinations thereof;
yellow pigments having the Color Index Numbers CI 11680, CI 11710, CI 15985, CI 19140, CI 20040, CI 21100, CI 21108, CI 47000, CI 47005, or combinations thereof;
green pigments having the Color Index Numbers CI 61565, CI 61570, CI 74260, or combinations thereof;
orange pigments having the Color Index Numbers CI 11725, CI 15510, CI 45370, CI 71105, or combinations thereof;
red pigments having the Color Index Numbers CI 12085, CI 12120, CI 12370, CI 12420, CI 12490, CI 14700, CI 15525, CI 15580, CI 15620, CI 15630, CI 15800, CI 15850, CI 15865, CI 15880, CI 17200, CI 26100, CI 45380, CI 45410, CI 58000, CI 73360, CI 73915, CI 75470; or combinations thereof; or combinations thereof.

10. The method according to claim 1, wherein the applying of the first and second compositions to the keratin material occurs by mixing the first composition and second composition together to form a mixture and then applying the mixture to the keratin material.

11. The method according to claim 1, wherein the first and second compositions (A) and (B) are applied sequentially to the keratin material.

12. The method according to claim 1, wherein the applying the heat treatment occurs by applying a hair dryer, a blow-dryer, a thermal hood, a straightening iron, a curling iron, an infrared lamp, or combinations thereof.

13. The method according to claim 1, wherein the applying the heat treatment occurs by applying a temperature ranging from 40° C. to 210° C. to the keratin material.

14. The method according to claim 1, further comprising applying a heat treatment to the keratin material prior to applying the first and second compositions.

15. The method according to claim 1, further comprising providing the first and second compositions; wherein the applying the first and second compositions comprises preparing a mixture of the first and second compositions, and applying the mixture to the keratin material for a predetermined amount of time; and further comprising optionally rinsing out the mixture before applying the heat treatment.

16. The method according to claim 1 wherein the applying of the heat treatment to the keratin material occurs after the first composition has been applied to the keratin material but before the second composition has been applied to the keratin material; and further comprising optionally rinsing out the first composition before applying the heat treatment and/or optionally rinsing out the second composition after the second composition is applied

17. The method according to claim 1, further comprising:
rinsing out the first composition before applying the second composition; and rinsing out the second composition before applying the heat treatment to the keratin material.
